SEQUENCE LISTING (8 sequences)

SEQ ID NO: 1 - DNA, 1569 bp, Mus musculus
  source 1..1569: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 2 - DNA, 1148 bp, Mus musculus
  source 1..1148: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgtcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 3 - DNA, 780 bp, Mus musculus
  source 1..780: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 4 - DNA, 388 bp, Mus musculus
  source 1..388: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 5 - protein, 313 aa, Homo sapiens
VKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 6 - DNA, 1626 bp, Homo sapiens
  source 1..1626: mol_type = other DNA
gctcagcatttggggacgctctcagctctcggcgcacggcccaggtaagcggggcgcgccctgcccgcccgcgatgggccgccagctagcggggtgtggagacgctgggaagaaggcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatca

SEQ ID NO: 7 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 8 - DNA, 3062 bp, Homo sapiens
  source 1..3062: mol_type = other DNA
agagctgagatcctacaggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttccaaaatcagagtggaatggttttgttatagatgctgtatcccactctttattcatgttcacattttaaaatcatttggaattctgcttcactcgcttaacatatacacaacacctgtaacatacaaggcaatgggctaggtgctccagaccgggaaaaggagggacaggaatgcttggtctgatgggctaatatggcatttagagaagtaccaaggtacagtggagccggtcacaaaagggcagacttgtagtagaattcagttgcaagagggattggggaatcttaaggaaaaaatagaatcttaaggaaaaaataactgggtgagacgtggactgtggacaggtgtggaaaaggcactctccatggaggtatgaatatgtagagggccaagagaggggagtacagggagaaatgagttgagcttgtctgaagtgaacttcaggaagaggaacataggctggaatttagattatgggggctctgaacaccaaactgagtttggacttaattgacttctg

(19) United States
(12) Patent Application Publication
(10) Pub. No.: US 2025/0242055 A1
(43) Pub. Date: Jul. 31, 2025

(54) Neuroprotection and Axon Regeneration Therapies for CNS Axonopathies by Modulating Membrane Structure, Cytoskeleton and Signaling Molecules

(71) Applicant: The Board of Trustees of the Leland Stanford Junior University, Stanford, CA (US)

(72) Inventors: Yang Hu, Menlo Park, CA (US); Liang Li, Hangzhou Zhejiang (CN); Xue Feng, Redwood City, CA (US); Haoliang Huang, Redwood City, CA (US)

(21) Appl. No.: 18/855,954

(22) PCT Filed: Apr. 6, 2023
(86) PCT No.: PCT/US2023/065474

§ 371 Date: Oct. 10, 2024

Related U.S. Application Data
(60) Provisional application No. 63/329,692, filed on Apr. 11, 2022.

(51) Int. Cl. A61K 48/00; A61K 38/17; A61K 38/49; C12N 15/113; C12N 15/864

(52) U.S. Cl. CPC A61K 48/005; A61K 38/177; A61K 38/49; C12N 15/113; C12N 15/8645; C12N 2750/14143

(57) ABSTRACT

Composition and methods are provided for the treatment of a mammalian subject for axonopathies by increasing activity of axon regeneration-associated gene (RAG) as identified herein, which include without limitation ANXA2, TPA, GSN, VIM, MPP1, ILK, ECM1, CALM1, AND ACAA2. These genes are shown to significantly promote axon regeneration, dramatically protects retinal ganglion cells and optic nerves, and preserve visual function in a clinically relevant model of glaucoma. A therapeutic entity may comprise, for example, a RAG protein, a gene therapy vector comprising a RAG coding sequence, a small molecule that enhances RAG activity, and the like.

### CROSS REFERENCE TO OTHER APPLICATIONS

This application claims the benefit of U.S. Provisional Application No. 63/329,692, filed Apr. 11, 2022, the contents of which are hereby incorporated by reference in its entirety.

### STATEMENT REGARDING FEDERALLY SPONSORED RESEARCH OR DEVELOPMENT

This invention was made with Government support under contract EY024932 awarded by the National Institutes of Health. The Government has certain rights in the invention.

### INCORPORATION BY REFERENCE LISTING PROVIDED AS A TEXT

A sequence listing is provided herewith as a sequence listing xml, “S22-079_STAN-1967WO_Seqlist” created on Apr. 6, 2023, and having a size of 17,261 Bytes. The contents of the sequence listing xml are incorporated by reference herein in their entirety.

### BACKGROUND

Axonopathy is a common early feature of central nervous system (CNS) neurodegenerative diseases, including glaucoma, which is characterized by optic nerve (ON) degeneration followed by progressive retinal ganglion cell (RGC) death, and is the leading cause of irreversible blindness. Glaucomatous neurodegeneration may be initiated by mechanical damage of the ON head due to elevated intraocular pressure (IOP). The axons of adult RGCs do not regenerate spontaneously after degeneration. Therefore, new neural repair therapies are desperately needed, especially because the only currently available treatments act by reducing IOP and fail to completely prevent the progression of glaucomatous neurodegeneration.

Deletion of Pten (phosphatase and tensin homolog) in RGCs remains by far the most potent single gene manipulation strategy to promote ON regeneration after ON crush (ONC) injury. Similar axon regeneration phenotypes after Pten deletion have been reported for mouse cortical motor neurons, drosophila sensory neurons, and C. elegans motor neurons. However, deregulated Pten activities have been implicated in diverse disorders, including metabolic diseases, tumor formation, cognitive impairment and even senescence. How to manipulate Pten-associated signaling molecules to maximize their axon regeneration activities while minimizing their deleterious side effects is extremely important for developing a clinically useful neural repair strategy. The prerequisite is to understand the specific downstream effectors of Pten in axon regeneration. Our previous studies elucidating the downstream signaling pathways of Pten in axon regeneration demonstrated that AKT coordinates positive signals from PI3K-PDK1 and negative signals from mTORC2 in regulating mTORC1 activation and GSK3β phosphorylation for ON regeneration. However, Pten deletion may also function through an AKT-independent pathway. The complicated crosstalk and feedback loops among Pten/PI3K/AKT/mTORC1/2 make it very difficult to pinpoint the key regeneration mediators that converge from these pathways. Another challenge is that the majority of Pten knockout (KO) RGCs cannot regenerate their axons even if they survive the injury, bulk RNA-seq of Pten KO RGCs without differentiation of regenerating RGCs from surviving but not regenerating RGCs is not very fruitful.

The axons of adult CNS neurons do not regenerate spontaneously after degeneration, which causes irreversible neuronal function deficits. Neural repair therapies that promote neuroprotection and axon regeneration are desperately needed.

### SUMMARY

Composition and methods are provided for the treatment of a mammalian subject for axonopathies by increasing activity of an axon regeneration-associated gene (RAG) that is identified herein, which RAG include without limitation ANXA2 (Annexin A2), tPA (tissue plasminogen activator), GSN (gelsolin), VIM (Vimentin), MPP1 (Membrane Palmitoylated Protein 1), ILK (Integrin Linked Kinase), extracellular matrix protein 1 (ECM1), calmodulin 1 (CALM1), and ACAA2 (Acetyl-CoA Acyltransferase 2). These genes are shown to significantly promote axon regeneration, dramatically protects retinal ganglion cells and optic nerves, and preserves visual function in a clinically relevant model of glaucoma. A therapeutic entity to increase RAG activity may comprise, for example, a RAG protein, a gene therapy vector comprising a RAG coding sequence, a small molecule that enhances RAG activity, and the like.

Compositions of the disclosure include, for example, a therapeutic gene therapy vector encoding a regeneration-associated gene (RAG) coding sequence operably linked to a neuron-specific promoter, which may be referred to herein as a therapeutic RAG vector; polynucleotide constructs and cells for producing such a therapeutic RAG vector, and virus particles comprising such a therapeutic RAG vector. RAGs of interest include e.g. one or more of ANXA2, TPA, GSN, VIM, MPP1, ILK, ECM1, CALM1, and ACAA2. In some embodiments a synergistic combination of ANXA2 and TPA sequences is provided as a therapeutic composition.

In some embodiments the RAG coding sequence is a human RAG coding sequence. In some embodiments the human RAG coding sequence encodes a variant with extended half-life. In some embodiments the vector is an adeno-associated virus or AAV vector. In some embodiments a virus particle comprising a therapeutic RAG vector is an adeno-associated virus (AAV). In some embodiments the neuron-specific promoter is selectively expressed in retinal ganglion cells (RGCs).

Methods are provided for reducing both neuronal cell body and axon death that results from axonopathies, the methods comprising contacting a neuron with an effective dose of the therapeutic RAG vector or an agent that increases activity of an RAG gene, e.g. a polypeptide, small molecule, etc. The contacting may be performed in vivo, e.g. on a human subject. In some embodiments the therapeutic RAG vector is administered as a virus particle formulation. In some embodiments the formulation is administered to an individual intravitreally for retina targeting. In some such embodiments the individual suffers from, or is at risk of developing, an optic nerve neuropathy, including without limitation, glaucoma.

In some embodiments a therapeutic formulation is provided, comprising a therapeutic RAG vector and a physiologically acceptable excipient. In some embodiments the vector is an AAV vector. In some embodiments a virus particle comprising a therapeutic RAG vector is an adenovirus-associated virus. In some embodiments the therapeutic formulation is provided in a unit dose, where a unit dose may comprise from about 10⁹ to about 10¹⁵ vector genomes/eye of the therapeutic RAG vector. The therapeutic formulation may be provided in a kit further comprising components for intravitreal administration, e.g. microcapillary needles, diluents, and the like.

Conditions for treatment include central and peripheral nervous systems axonopathies, particularly conditions involving Wallerian degeneration. The axonopathy may be the result of disease or trauma, such as CNS axonapathies amyotrophic lateral sclerosis (ALS) and hereditary spastic paraplegia (HSP), PNS nerve injury, traumatic brain injury, spinal cord injury or neuronal injury induced by a toxic agent such as a chemotherapeutic agent. In one embodiment, the axonopathy is a neuronal injury induced by a chemotherapeutic agent, e.g. a taxane, vincristine, etc. In some embodiments the axonopathy is an optic nerve neuropathy. In some embodiments the optic nerve neuropathy is glaucoma, e.g. open-angle glaucoma, angle-closure glaucoma, etc. In other embodiments an optic neuropathy is non-arteritic ischemic optic neuropathy (NAION), optic neuritis, ischemic optic neuropathy, inflammatory (non-demyelinating) and traumatic optic neuropathy, etc.

In another embodiment, methods are provided for screening RGCs to identify regenerative factors. The methods definitively label and purify regenerating and non-regenerating RGCs separately. Because both sets of RGCs undergo the same genetic manipulation and injury and differ only in axon regeneration capability, comparison of gene expression in these two populations provides an informative way to find genes that are truly associated with axon regeneration. Single cell RNA sequencing identifies differentially expressed genes. The sequencing may be performed with plate-based Smart-Seq2, which allows sensitivity and accuracy in the detection of genes that are differentially expressed among regenerating and non-regenerating RGCs.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1K. Retrograde tracing of regenerating RGCs by intraorbital ON dye injection. (A) The intraorbital portion of ON (˜2 mm) was exposed by pushing through the ocular muscles and soft tissues under the conjunctiva, without injuring the retro-orbital sinus. The ONC site is about 0.5 mm from the eyeball, leaving 1-1.5 mm distal portion for dye injection. (B) Confocal images of retinal wholemounts of naïve mice one day after dextran-FITC intraorbital ON injection, showing co-localization of the dextran and pan-RGC marker (RBPMS)-labeled RGCs, which are distinct from AP2α⁺ amacrine cells. Scale bar, 50 μm. (C, D, E) Cartoon illustration of intravitreal injection of anterograde tracer dye CTB-555 and intraorbital ON injection of retrograde tracer dye dextran-FITC in naïve, ONC injured WT and Pten KO mice at 14dpc; and corresponding fluorescent and bright field (BF) images of ONs showing the labeled axons and ONC sites. (F, G, H) SLO retinal fundus images of live animals showing the dextran-FITC labeled RGCs in naïve, ONC injured WT and Pten KO mice at 14dpc. (I, J, K) Retinal wholemounts of naïve, ONC injured WT, and Pten KO mice at 14dpc showing dextran-FITC labeled RGCs. Scale bar, upper panel: 500 μm; lower panel: 50 μm.

FIGS. 2A-2H. Regenerating RGCs (regRGCs) and surviving but not regenerating RGCs (surRGCs): labeling, isolation, and Smart-Seq2 scRNA-seq. (A) Timeline of experimental design for Pten deletion in RGCs 2 weeks before ONC, SC retrograde labeling of naïve RGCs, ON retrograde tracing regRGCs, and tissue collection at 14dpc. (B) Cartoon illustration of SC injection of retrobeads-488 and intraorbital ON injection of retrograde tracer dye dextran-Red; and SLO retinal fundus image of live animal showing RGCs labeled with retrobeads and retinal wholemounts showing surRGCs (green only) and regRGCs (red or yellow), in Pten KO mice at 14dpc. Scale bar, 500 μm in the whole retina, 50 μm in the zoom-in. (C) Quantification of the numbers of regRGCs/retina labeled by our tracing strategy in WT and Pten KO mice at 14dpc. n=6. All the data are presented as means±s.e.m, *** p<0.001, two-tailed student t test. (D) FACS gating strategy for surRGCs (green only, no red) and regRGCs (red alone or red/green double) purification. (E) Smart-Seq2 preparation for scRNA-seq. (F) Quality control of scRNA-seq data showing number of genes detected/cell (nFeature), total reads/cell (nCount), and percentage of mitochondria genes (percent). (G) UMAP (Uniform Manifold Approximation and Projection) visualization of the transcriptional heterogeneity of 630 RGCs (340 regRGCs and 290 surRGCs) isolated from Pten KO mice at 14dpc. Cells are colored into 5 clusters. (H) The 5 RGC clusters are superimposed with regeneration identify: surRGCs are in green and regRGCs are in red.

FIGS. 3A-3F. Comparison of Smart-Seq2 transcriptomes of regRGCs and surRGCs reveals biological pathways and DEGs associated with RGC regeneration and survival. (A, B) Top 15 enriched GO-pathways associated with regRGCs (A) or surRGCs (B). The size of each circle represents the numbers of genes enriched in each pathway, and the color represents the adjusted p value. (C, D) Cnetplot showing the interaction of 5 enriched pathways and their associated genes in regRGCs (C) and surRGCs (D). The size of each circle associated with each pathway represents the numbers of genes enriched in each pathway, and the color of each gene represents the fold change (FC). (E) VolcanoPlot of DEGs of regRGCs. Adjusted p value <0.05, avg_log 2FC >0.25. The red genes are upregulated, and the green genes are downregulated in regRGCs. The grey genes are not significantly changed. (F) Dotplot showing expression of indicated regeneration-associated genes in regRGCs and surRGCs. The size of each circle represents the percentage of RGCs expressing the gene, and the color represents the expression level.

FIGS. 4A-4C. Anxa2 is the most potent of the 6 regeneration-associated genes that promote significant axon regeneration after ONC injury. (A) AAV vector used for driving transgene expression under mSncg promoter and the timeline of experimental design for AAV-mediated transgene expression in RGCs 2 weeks before ONC and intravitreal anterograde tracing of regenerating axons in ONs at 14dpc. (B) Confocal images of ON wholemounts after optical clearance showing maximum intensity projection of regenerating fibers labeled with CTB-Alexa 555 at 14dpc. Scale bar, 100 μm. *: crush site. (C) Quantification of regenerating fibers at different distances distal to the lesion site. Data are presented as means±s.e.m, n=8-10. *: p<0.05, **: p<0.01, ***: p<0.001, ****: p<0.0001, two-way ANOVA with Sidak's multiple comparisons test.

FIGS. 5A-5D. Anxa2 and tPA act synergistically with Pten deletion to promote potent lengthy axon regeneration. (A) Confocal images of ON wholemounts after optical clearance showing maximum intensity projection of regenerating fibers labeled with CTB-Alexa 555 at 14dpc. Scale bar, 500 μm. *: crush site. (B) Quantification of regenerating fibers at different distances distal to the lesion site. Data are presented as means±s.e.m, n=8. *: p<0.05, **: p<0.01, two-way ANOVA with Sidak's multiple comparisons test. (C) iDISCO clearance of whole brain with attached ONs. (D) Light-sheet fluorescent images of regenerating axons in ON, optic chiasm, and optic tract. Scale bar, 500 μm.

FIGS. 6A-6G. RGC-specific Anxa2 overexpression significantly promotes survival of both RGC somata and axons, and preserves visual functions in SOHU glaucoma model. (A) Representative OCT images of mouse retinas from SOHU glaucomatous eyes and contralateral control (CL) eyes at 3 weeks post SO injection (3wpi). GCC: ganglion cell complex, including RNFL, GCL and IPL layers; indicated as double end arrows. Quantification of GCC thickness measured by OCT at 3wpi, represented as percentage of GCC thickness in the SOHU eyes compared to the sham CL eyes. (B) Upper panel, representative confocal images of the whole flat-mounted retinas showing surviving RBPMS-positive (red) RGCs at 3wpi, Scale bar, 500 μm. Lower panel, representative confocal images of peripheral flat-mounted retinas showing surviving RBPMS-labeled RGCs at 3wpi, Scale bar, 50 μm. (C) Quantification of surviving RGC somata in peripheral, middle, and central retinas at 3wpi, represented as percentage of glaucomatous eyes compared to the sham CL eyes. (D) Light microscope images of semi-thin transverse sections of ON with PPD staining at 3wpi. Scale bar, 10 μm. (E) Quantification of surviving axons at 3wpi, represented as percentage of glaucomatous eyes compared to the sham CL eyes. (F) Visual acuity measured by OKR at 3wpi, represented as percentage of glaucomatous eyes compared to the sham CL eyes. (G) Left: representative wave forms of PERG at baseline and 3wpi, blue traces represent glaucomatous eyes, black traces represent contralateral control (CL) eyes. Right: quantification of P1-N2 amplitude of PERG at 3wpi, represented as a percentage of glaucomatous eyes compared to the sham CL eyes. All the quantification, data are presented as means±s.e.m, n=13-16 in each group, *: p<0.05, **: p<0.01, ***: p<0.001, one-way ANOVA with Tukey's multiple comparisons test.

FIGS. 7A-7C. Testing retrograde tracing dyes for RGC labeling. (A) Intraorbital ON injection of retrograde tracer dyes in naïve mouse; and retinal wholemounts showing the labeled RGCs. (B) ON wholemounts showing injection site of dextran at 1 or 1.5 mm distal to crush site in Pten KO mice; the SLO retinal fundus images and retinal wholemounts showing labeled regenerating RGCs. (C) SC injection of retrograde tracer dyes in naïve mouse; and retinal wholemounts showing RGCs labeled with retrograde dye 4 weeks after SC injection.

FIGS. 8A-8C. DEGs and GO biological pathways enriched in each cluster. (A) Heatmap showing top 10 genes selectively expressed in each cluster in all the RGCs. (B) Top 15 enriched GO-pathways analyzed with DEGs of each cluster. The size of each circle represents the numbers of genes enriched in each pathway, and the color represents the adjusted p value. (C) Dotplot showing expression of RGC subtype-marker genes defined in a previous study (Tran, et al, 2019). The size of each circle represents the percentage of RGCs expressing the gene, and the color represents the expression level.

FIGS. 9A-9C. The expression of selective regeneration-associated DEGs. (A) Violin plots of the expression of selective regeneration-associated DEGs in regRGCs and surRGCs. (B) The expression of selective regeneration-associated DEGs in the 45 subtypes of naïve adult mouse RGCs according to online database. (C) The expression of selective regeneration-associated DEGs in the 45 subtypes of adult mouse RGCs at 14dpc according to online database.

FIGS. 10A-10C. The AAV-mediated expression of selective regeneration-associated genes in RGCs. (A) AAV-mSncg promoter-mediated transgene expression in RGCs labeled by HA antibodies 2 weeks after intravitreal injection. Scale bar, 50 μm. (B) Confocal images of retinal wholemounts showing RBPMS+ RGCs at 14dpc. Scale bar, 50 μm. (C) Quantification of surviving RGC somata in peripheral retina at 14dpc, represented as percentage of crushed eyes compared to the sham CL eyes. Data are presented as means±s.e.m, n=5-7 in each group. *: p<0.05, one-way ANOVA with Tukey's multiple comparisons test.

FIGS. 11A-11B. ILK acts downstream of Anxa2 in axon regeneration. (A) Confocal images of ON wholemounts after optical clearance showing maximum intensity projection of regenerating fibers labeled with CTB-Alexa 555 at 14dpc. Scale bar, 100 μm. *: crush site. (B) Quantification of regenerating fibers at different distances distal to the lesion site. Green *: comparison of ILK-CA and Ctrl; red *: comparison of ILK-WT and Ctrl. Data are presented as means±s.e.m, n=8-10. *: p<0.05, **: p<0.01, two-way ANOVA with Sidak's multiple comparisons test.

FIGS. 12A-12B. Anxa2 and tPA act synergistically with Pten deletion to promote RGC survival after ONC. (A) Confocal images of retinal wholemounts showing RBPMS+ RGCs and HA labeling of Anxa2 at 14dpc. Scale bar, left panel: 500 μm; right panel: 50 μm. (B) Quantification of surviving RGCs somata in peripheral retina at 14dpc, represented as percentage of crushed eyes compared to the sham CL eyes. Data are presented as means±s.e.m, n=6-11 in each group. **: p<0.01, ***: p<0.001, ****: p<0.0001, one-way ANOVA with Tukey's multiple comparisons test.

FIGS. 13A-13C. The SOHU glaucoma model. (A) The timeline of AAV intravitreal injection and SO intracameral injection to generate the SOHU glaucoma model. (B) Photos of mouse eyeballs with or without SO intracameral injection and correlated cartoon illustration and anterior segment OCT live images. (C) IOP of naïve and SOHU eyes at 3wpi. Data are presented as means±s.e.m, n=13-16 in each group. ****: p<0.0001, one-way ANOVA with Tukey's multiple comparisons test.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Annexin A2 (ANXA2), is involved in diverse cellular processes such as cell motility (especially that of the epithelial cells), linkage of membrane-associated protein complexes to the actin cytoskeleton, endocytosis, fibrinolysis, ion channel formation, and cell matrix interactions. It is a calcium-dependent phospholipid-binding protein whose function is to help organize exocytosis of intracellular proteins to the extracellular domain. Reference sequences for the human mRNA and protein may be found at Genbank, accession numbers: NM_001002857, NM_001002858, NM_001136015, NM_004039; and NP_001002857, NP_001002858, NP_001129487, NP_004030.

Tissue plasminogen activator (tPA) is a serine protease (EC 3.4.21.68) found on endothelial cells, that line the blood vessels. As an enzyme, it catalyzes the conversion of plasminogen to plasmin, the major enzyme responsible for clot breakdown. tPA is used in some cases of diseases that feature blood clots, such as pulmonary embolism, myocardial infarction, and stroke. The most common use is for ischemic stroke. Reference sequences for the human mRNA and protein may be found at Genbank, accession numbers: NM_033011, NM_000930, NM_000931, NM_001319189; and NP_000921, NP_001306118, NP_127509.

Gelsolin (GSN) is an actin-binding protein that is a key regulator of actin filament assembly and disassembly. Gelsolin is one of the most potent members of the actin-severing gelsolin/villin superfamily, as it severs with nearly 100% efficiency. Cellular gelsolin, found within the cytosol and mitochondria, has a closely related secreted form, plasma gelsolin, that contains an additional 24 AA N-terminal extension. Plasma gelsolin's ability to sever actin filaments helps the body recover from disease and injury that leaks cellular actin into the blood. Additionally it plays important roles in host innate immunity, activating macrophages and localizing of inflammation.

Gelsolin is an 82-kD protein with six homologous subdomains, referred to as S1-S6. Each subdomain is composed of a five-stranded β-sheet, flanked by two α-helices, one positioned perpendicular with respect to the strands and one positioned parallel. The β-sheets of the three N-terminal subdomains (S1-S3) join to form an extended β-sheet, as do the β-sheets of the C-terminal subdomains (S4-S6).

Gelsolin's activity is stimulated by calcium ions (Ca2+). Although the protein retains its overall structural integrity in both activated and deactivated states, the S6 helical tail moves like a latch depending on the concentration of calcium ions. The C-terminal end detects the calcium concentration within the cell. When there is no Ca2+ present, the tail of S6 shields the actin-binding sites on one of S2's helices. When a calcium ion attaches to the S6 tail, however, it straightens, exposing the S2 actin-binding sites. The N-terminal is directly involved in the severing of actin. S2 and S3 bind to the actin before the binding of S1 severs actin-actin bonds and caps the barbed end.

Reference sequences for the human mRNA and protein may be found at Genbank, accession numbers: NM_001127662, NM_001127663, NM_001127664, NM_001127665; and NP_001121134, NP_001121135, NP_001121136, NP_001121137.

Vimentin (VIM) is a type III intermediate filament (IF) protein that is expressed in mesenchymal cells. IF proteins are found in all animal cells as well as bacteria. Intermediate filaments, along with tubulin-based microtubules and actin-based microfilaments, comprises the cytoskeleton. All IF proteins are expressed in a highly developmentally-regulated fashion; vimentin is the major cytoskeletal component of mesenchymal cells. Because of this, vimentin is often used as a marker of mesenchymally-derived cells or cells undergoing an epithelial-to-mesenchymal transition (EMT) during both normal development and metastatic progression.

A vimentin monomer, like all other intermediate filaments, has a central α-helical domain, capped on each end by non-helical amino (head) and carboxyl (tail) domains. Two monomers are likely co-translationally expressed in a way that facilitates their formation of a coiled-coil dimer, which is the basic subunit of vimentin assembly.

The α-helical sequences contain a pattern of hydrophobic amino acids that contribute to forming a “hydrophobic seal” on the surface of the helix. In addition, there is a periodic distribution of acidic and basic amino acids that seems to play an important role in stabilizing coiled-coil dimers. The spacing of the charged residues is optimal for ionic salt bridges, which allows for the stabilization of the α-helix structure. While this type of stabilization is intuitive for intrachain interactions, rather than interchain interactions, scientists have proposed that perhaps the switch from intrachain salt bridges formed by acidic and basic residues to the interchain ionic associations contributes to the assembly of the filament.

Reference sequences for the human mRNA and protein may be found at Genbank, accession numbers: NM_003380; and NP_003371.

55 kDa erythrocyte membrane protein (MPP1) is the prototype of a family of membrane-associated proteins termed MAGUKs (membrane-associated guanylate kinase homologs). MAGUKs interact with the cytoskeleton and regulate cell proliferation, signaling pathways, and intracellular junctions. Palmitoylated membrane protein 1 contains a conserved sequence, called the SH3 (src homology 3) motif, found in several other proteins that associate with the cytoskeleton and are suspected to play important roles in signal transduction.

Reference sequences for the human mRNA and protein may be found at Genbank, accession numbers: NM_001166460, NM_001166461, NM_001166462, NM_002436; and NP_001159932, NP_001159933, NP_001159934, NP_002427.

Integrin-linked kinase (ILK) are a subfamily of Raf-like kinases (RAF). The structure of ILK consists of three features: 5 ankyrin repeats in the N-terminus, phosphoinositide binding motif and extreme N-terminus of kinase catalytic domain. Integrins lack enzymatic activity and depend on adapters to signal proteins. ILK is linked to beta-1 and beta-3 integrin cytoplasmic domains and is one of the best described integrins.

Transduction of extracellular matrix signals through integrins influences intracellular and extracellular functions, and appears to require interaction of integrin cytoplasmic domains with cellular proteins. Integrin-linked kinase (ILK), interacts with the cytoplasmic domain of beta-1 integrin. Multiple alternatively spliced transcript variants encoding the same protein have been found for this gene. Recent results showed that the C-terminal kinase domain is actually a pseudo-kinase with adaptor function.

Reference sequences for the human mRNA and protein may be found at Genbank, accession numbers: NM_001014794, NM_001014795, NM_001278441, NM_001278442, NM_004517; and NP_001014794, NP_001014795, NP_001265370, NP_001265371, NP 004508.

Extracellular Matrix Protein 1 (ECM1) is a soluble protein that is involved in endochondral bone formation, angiogenesis, and tumor biology. It also interacts with a variety of extracellular and structural proteins, contributing to the maintenance of skin integrity and homeostasis. Mutations in this gene are associated with lipoid proteinosis disorder (also known as hyalinosis cutis et mucosae or Urbach-Wiethe disease) that is characterized by generalized thickening of skin, mucosae and certain viscera. ECM1 stimulates the proliferation of endothelial cells and promotes angiogenesis, and inhibits MMP9 proteolytic activity.

Calmodulin 1 (CALM1) belongs to the members of the EF-hand calcium-binding protein family. Calcium-induced activation of calmodulin regulates and modulates the function of cardiac ion channels. Calmodulin acts as part of a calcium signal transduction pathway by mediating the control of a large number of enzymes, ion channels, aquaporins and other proteins through calcium-binding. Calcium-binding is required for the activation of calmodulin. Among the enzymes to be stimulated by the calmodulin-calcium complex are a number of protein kinases, such as myosin light-chain kinases and calmodulin-dependent protein kinase type II (CaMK2), and phosphatases. Together with CCP110 and centrin, is involved in a genetic pathway that regulates the centrosome cycle and progression through cytokinesis. Mediates calcium-dependent inactivation of CACNA1C. Positively regulates calcium-activated potassium channel activity of KCNN2. Forms a potassium channel complex with KCNQ1 and regulates electrophysiological activity of the channel via calcium-binding. Acts as a sensor to modulate the endoplasmic reticulum contacts with other organelles mediated by VMP1:ATP2A2.

3-Ketoacyl-CoA thiolase, also known as acetyl-Coenzyme A acyltransferase 2 (ACAA2) is 41.9 kDa protein that catalyzes the last step of the mitochondrial fatty acid beta oxidation spiral. Unlike most mitochondrial matrix proteins, it contains a non-cleavable amino-terminal targeting signal. ACAA2 has been shown to be a functional BNIP3 binding partner, which provides a possible link between fatty acid metabolism and cell apoptosis.

Reference sequences for the human mRNA and protein may be found at Genbank, accession numbers: NM_006111; and NP_006102.

AAV gene therapy. Utilizing a viral vehicle to deliver genetic material into cells allows direct targeting of pathogenic molecules and restoration of function. The retina is an advantageous target for gene therapy due to its easy access, confined non-systemic localization, partial immune privilege, and well-established definitive functional readouts. The success of adeno-associated virus (AAV)-mediated gene replacement in treating inherited retinal disease makes RGC-specific therapy with AAV a promising gene therapy strategy for optic neuropathies. Because AAV is non-pathogenic and cannot reproduce itself without helper viruses, it has served as a primary vehicle for gene therapy. It is a single-stranded DNA virus that stably and efficiently infects a wide variety of cells in multiple tissues. AAV2, the best-characterized AAV serotype, efficiently infects RGCs in retina after intravitreal injection.

In some embodiments, the vector is a recombinant adeno-associated virus (AAV) vector. AAV vectors are DNA viruses of relatively small size that can integrate, in a stable and site specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV can infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Viral., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Rudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Rudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCTIUS2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g. AAV virus particle including, but not limited to, AAV1, AAV2, AAV3, AAV4, AAV5, AAV6, AAV7, AAV8, AAV9, AAVIO, AAVII, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

A neuron-specific promoter allows precise manipulation of gene expression without affecting other cell types. Aspects of the present invention encompass expression cassettes and/or vectors comprising polynucleotide sequences of interest for expression in targeted cells. The polynucleotides can comprise promoters operably linked to RAG coding sequence. Targeted expression is accomplished using a cell-selective or cell-specific promoter. Examples are promoters for somatostatin, parvalbumin, GABAa6, L7, and calbindin. Other cell specific promoters can be promoters for kinases such as PKC, PKA, and CaMKII; promoters for other ligand receptors such as NMDAR1, NNIDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Promoters of particular interest are RGC specific promoters, e.g. murine γ-synuclein (mSncg) promoter, which drives specific, potent and sustained transgene expression in rodent RGCs, nonhuman primate RGCs, and human primary RGCs, as well as human induced Pluripotent Stem Cell (IPS) stem cell-derived RGCs.

In some embodiments a promoter is used for the selective expression of an operably linked gene in retinal ganglion cells (RGCs). In some embodiments the promoter comprises or consists of an mSncg promoter, see for example U.S. provisional application 63/284,424, herein specifically incorporated by reference. In some embodiments the promoter sequence is provided in the context of a vector for expression, including without limitation a viral vector, e.g. an AAV vector. Cells of interest for expression include, without limitation, cells in the eye and progenitors thereof, e.g. retinal cells, particularly retinal ganglion cells, and their progenitors.

As used herein, the term “wildtype” generally refers to a gene, or sub-portion thereof, in the subject that is not mutated, or not substantially mutated (e.g., at either allele) so as to affect the function of the gene. Accordingly, a wildtype locus may contain the common (i.e., most prevalent, normal, etc.) sequence of the gene, or essentially the common sequence of the gene, without mutation, or without substantial mutation, affecting the function of the gene. The “common sequence”, as used in this context, generally refers to the gene sequence as it most frequently occurs in a natural population. In some instances, common sequences may be represented by a reference sequence, e.g., a reference sequence as it appears in a sequence database, such as but not limited to e.g., GenBank database (NCBI), UniProt database (EBI/SIB/PIR), or the like. In some instances, a wildtype locus may be identical or substantially identical to a reference sequence.

By “treatment” it is meant that at least an amelioration of one or more symptoms associated with a neurodegenerative disorder afflicting the subject is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

“Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs (e.g., the optic nerve (ON)), etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The term ‘neuroprotective’ as used herein refers to the ability to protect neurons or their axons or synapses in the central or peripheral nervous system from damage or death. Many different types of insult can lead to neuronal damage or death, for example: metabolic stress caused by hypoxia, hypoglycemia, diabetes, loss of ionic homeostasis or other deleterious process, physical injury of neurons, exposure to toxic agents and numerous diseases affecting the nervous system including inherited disorders. The presence of an agent that is neuroprotective enables a neuron to remain viable upon exposure to insults that would otherwise cause a loss of functional integrity in an unprotected neuron.

Axonopathy, broadly defined as functional or structural defects in the axon or its terminal, has been established as a major early contributor to the genesis, progression, and symptomology of neurodegenerative disorders. Axon degeneration is an active process, as demonstrated in Wallerian degeneration, which involves the fragmentation and disintegration of an axon distal to the site of an injury. Axonopathy is often considered in the context of peripheral motor and sensory neurons, given their length, the presence of diseases that specifically affect these systems, and their sensitivity to challenges such as chemotherapy drugs or metabolic disorders such as diabetes. However, these characteristics are not limited to the peripheral nervous system. Glaucoma, a neuropathy affecting axons of the optic nerve, one of the few central nervous system components outside of the brain and spinal cord. Glaucoma shares commonalities with other central neurodegenerations such as amyotrophic lateral sclerosis (ALS) and hereditary spastic paraplegia (HSP), Alzheimer's, Parkinson's, and Huntington's diseases, often exhibiting comorbidity with those conditions, as well as exhibiting similar mechanisms with these and other axonopathies.

In one embodiment a therapeutic vector comprising a RAG coding sequence is intended for use as a neuroprotective medicament in the treatment of a neurodegenerative disorder resulting from neuronal injury. The term ‘injury’ as used herein refers to damage inflicted on the neuron, whether in the cell body or in axonal or dendritic processes. This can be a physical injury in the conventional sense i.e. traumatic injury to the brain, spinal cord or peripheral nerves caused by an external force applied to a subject. Other damaging external factors are for example environmental toxins such as mercury and other heavy metals, pesticides and solvents. Alternatively, injury can result from an insult to the neuron originating from within the subject, for example: reduced oxygen and energy supply as in ischemic stroke and diabetic neuropathy, autoimmune attack as in multiple sclerosis or oxidative stress and free-radical generation as is believed to be important in amyotrophic lateral sclerosis. Injury is also used here to refer to any defect in the mechanism of axonal transport.

In another embodiment, a therapeutic vector comprising a RAG coding sequence is intended for use as a neuroprotective medicament wherein the neurodegenerative disorder is caused by a neuronal injury resulting from a disease. In some instances, the optic neuropathy and/or neurodegenerative disorder treated according to the methods described herein may be an optic neuropathy such as Leber's hereditary optic neuropathy (LHON), Anterior ischemic optic neuropathy (AION), optic disc drusen (ODD), dominant optic atrophy (DOA), ON damage associated with glaucoma, or other CNS neurodegenerative disorder leading to ON degeneration. In some instances of the methods disclosed herein, the disease or disorder may involve inflammation leading to degeneration of the ON.

In one embodiment, the neurodegenerative disorder is an ophthalmic disorder such as glaucoma. Glaucomas are a group of eye disorders characterized by progressive optic nerve damage in which an important part is a relative increase in intraocular pressure (IOP) that can lead to irreversible loss of vision. Glaucomas are categorized as open-angle glaucoma or angle-closure glaucoma. The “angle” refers to the angle formed by the junction of the iris and cornea at the periphery of the anterior chamber. The angle is where >98% of the aqueous humor exits the eye via either the trabecular meshwork and the Schlemm canal or the ciliary body face and choroidal vasculature. Glaucomas are further subdivided into primary (cause of outflow resistance or angle closure is unknown) and secondary (outflow resistance results from a known disorder), accounting for >20 adult types. Another group of glaucoma patients does not have IOP elevation, which in general is called normal tension glaucoma (NTG). NTG is also associated with progressive optic nerve degeneration and RGC death. Thus they are also subject to this gene therapy treatment.

Axons of retinal ganglion cells travel through the optic nerve carrying visual information from the eye to the brain. Damage to these axons causes ganglion cell death with resultant optic nerve atrophy and patchy vision loss. Elevated intraocular pressure (IOP; in unaffected eyes, the average range is 11 to 21 mm Hg) plays a role in axonal damage, either by direct nerve compression or diminution of blood flow. However, the relationship between externally measured pressure and nerve damage is complicated. Of people with IOP >21 mm Hg (ie, ocular hypertension), only about 1 to 2%/year (about 10% over 5 years) develop glaucoma. Additionally, about one third of patients with glaucoma do not have IOP >21 mm Hg (known as low-tension glaucoma or normal-tension glaucoma).

IOP is determined by the balance of aqueous secretion and drainage. Elevated IOP is caused by inhibited or obstructed outflow, not oversecretion; a combination of factors in the trabecular meshwork (eg, dysregulation of extracellular matrix, cytoskeletal abnormalities) appear to be involved. In open-angle glaucoma, IOP is elevated because outflow is inadequate despite an angle that appears unobstructed. In angle-closure glaucoma, IOP is elevated when a physical distortion of the peripheral iris mechanically blocks outflow.

Symptoms and signs of glaucoma vary with the type of glaucoma, but the defining characteristic is optic nerve damage as evidenced by an abnormal optic disk and certain types of visual field deficits. Glaucoma is diagnosed when characteristic findings of optic nerve damage are present and other causes have been excluded. Elevated IOP makes the diagnosis more likely, but elevated IOP can occur in the absence of glaucoma and is not essential for making the diagnosis.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) after the administration of a second therapeutic agent.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes lipoproteins, glycoproteins, and the like.

A “host cell,” as used herein, denotes an in vivo or in vitro eukaryotic cell, or a cell from a multicellular organism (e.g., a cell line) cultured as a unicellular entity, which eukaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector, a guide RNA, a donor DNA template, and the like. For example, a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

### METHODS

As summarized above, aspects of the instant disclosure include methods of treating a subject for an axonopathy. A variety of neurodegenerative disorders also may be treated by practice of the methods described herein, particularly glaucoma, e.g. open-angle glaucoma or angle-closure glaucoma. In some embodiments, provided herein is a method of treating an individual suffering from an optic nerve (ON) axonopathy, comprising intravitreally administering a RAG agent into the subject, thereby treating the ON axonopathy. In some embodiments, provided herein is a method of reducing or ameliorating degeneration of axons and/or soma of RGCs, comprising intravitreally administering a RAG agent into a mammalian subject experiencing or at imminent risk of an ON axonopathy.

In some aspects, provided herein is a method of inducing neuroprotection/increasing survival/promoting functional recovery of RGC somata and axons, comprising intravitreally administering a RAG agent into a mammalian subject experiencing or at risk of an ON axonopathy. In some embodiments of the method, the ON neuropathy is retinal ganglion cell degeneration, including glaucoma, optic neuritis, ON traumatic injury and other ON-related diseases. In some embodiments the individual has been diagnosed with the ON neuropathy prior to treatment.

In some embodiments, the therapeutic vector comprises an AAV vector, which comprises a murine γ-synuclein promoter in operable linkage with a nucleic acid encoding a human or murine RAG protein.

Various subjects may be treated in the methods of the present disclosure. In some instances, treated subjects may be mammals, including but not limited to e.g., rodents (e.g., rats, mice, etc.), non-human primates (e.g., macaques, marmosets, tamarins, spider monkeys, owl monkeys, vervet monkeys, squirrel monkeys, baboons, chimpanzees, etc.), humans, and the like. In some instances, a treated subject may be an animal model (e.g., a rodent model, a non-human primate model, etc.) of an optic neuropathy and/or neurodegenerative disorder. In some instances, a treated subject may be a human subject, including but not limited to e.g., a human subject having an optic neuropathy and/or neurodegenerative disorder, a human subject at increased risk of developing an optic neuropathy and/or neurodegenerative disorder, a human subject carrying an RAG mutation that is causative to a disease, a human subject with low NAD level in neurons, a human subject of advanced age (e.g., at least 60 years of age, at least 65 years of age, at least 70 years of age, at least 75 years of age, at least 80 years of age, at least 85 years of age, at least 90 years of age, etc.), or a combination thereof. Treated subjects may or may not be symptomatic, e.g., subject may or may not display or have previously displayed one or more symptoms of an optic neuropathy and/or neurodegenerative disorder, including but not limited to e.g., those optic neuropathies and/or neurodegenerative disorders described herein.

Methods of the present disclosure may include administering to a subject a therapeutic vector that targets RGCs and reduces RGC and optic nerve degeneration or a polynucleotide encoding a RAG sequence where the protein shares 100% sequence identity or less than 100% sequence identity, including e.g., at least 99%, at least 98%, at least 97% at least 96%, at least 95%, at least 90%, at least 85%, at least 80%, etc., sequence identity, with a protein or amino acid sequence of a protein described herein. In some instances, inducers may include a polynucleotide encoding the protein, or a fragment thereof, including where the polynucleotide shares 100% sequence identity or less than 100% sequence identity, including e.g., at least 99%, at least 98%, at least 97% at least 96%, at least 95%, at least 90%, at least 85%, at least 80%, etc., sequence identity, with an encoding polynucleotide identified herein.

Administration of an agent to a subject, as described herein, may be performed employing various routes of administration. The route of administration may be selected according to a variety of factors including, but not necessarily limited to, the condition to be treated, the formulation and/or device used, the patient to be treated, and the like. Routes of administration useful in the disclosed methods include but are not limited to intravitreal injection, oral and parenteral routes, such as intravenous (iv), intraperitoneal (ip), rectal, topical, ophthalmic, nasal, and transdermal. Formulations for these dosage forms are described herein.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

In those embodiments where an effective amount of an active agent is administered to the subject, the amount or dosage is effective when administered for a suitable period of time, such as one week or longer, including two weeks or longer, such as 3 weeks or longer, 4 weeks or longer, 8 weeks or longer, etc., so as to evidence a reduction in the disorder, e.g., a reduction in a symptom of the disorder or in a marker of disease pathology. For example, an effective dose is the dose that, when administered for a suitable period of time, such as at least about one week, and maybe about two weeks, or more, up to a period of about 3 weeks, 4 weeks, 8 weeks, or longer, will reduce a symptom of the disorder, for example, by about 10% or more, by about 20% or more, e.g., by 30% or more, by 40% or more, or by 50% or more, in some instances by 60% or more, by 70% or more, by 80% or more, or by 90% or more, for example, and will halt progression of the disorder in the subject. In some instances, an effective amount or dose of active agent will not only slow or halt the progression of the disease condition but will also induce the reversal of the condition, i.e., will cause an improvement in the neurological health of the subject. For example, in some instances, an effective amount is the amount that when administered for a suitable period of time, for example, at least about one week, and/or about two weeks, or more, up to a period of about 3 weeks, 4 weeks, 8 weeks, or longer will improve, stabilize, or at least reduce the progression of a disorder in subject, for example 1.5-fold, 2-fold, 3-fold, 4-fold, 5-fold, in some instances 6-fold, 7-fold, 8-fold, 9-fold, or 10-fold or more relative to the subject's condition prior to administration.

In some instances, in those embodiments where an effective amount of an active agent is administered to the subject, the amount or dosage is effective when administered for a suitable period of time to result in a reduction in RGC degeneration in the subject. Such a reduction may manifest in various ways, including but not limited to e.g., an increase in the number, size or length of RGCs, or a reduction in the amount of degeneration of RGCs, or their axons or soma, or the like. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., reduction in RGC degeneration. In some instances, methods of the present disclosure may result in at least a 5%, e.g., at least a 10%, at least a 15%, at least a 20%, at least a 25%, at least a 30%, at least a 35%, at least a 40%, at least a 45%, at least a 50%, at least a 55%, at least a 60%, at least a 65%, at least a 70% at least a 75%, at least a 80%, e.g., increase in RGC number, size or length of RGC axons or somata. Various methods of assessing the amount of RGC degeneration or increase in number, size or length of RGC axons or somata may be employed, including invasive and non-invasive techniques, such as electrophysiology measurement for RGC neuronal function, visual acuity, OCT imaging, fundus imaging, histology studies of RGC somata and axons morphology.

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of an agent is an amount that is sufficient, when administered to the individual, to palliate, ameliorate, stabilize, reverse, prevent, slow or delay the progression of the disease state (e.g., neurodegeneration) by, for example, inhibiting gene expression product formation, or otherwise preventing the symptoms or clinical progression of a neurodegenerative disorder present in the subject.

In some embodiments a therapeutic dose is determined by the number of vector genomes administered to a retina, e.g. at least about 10⁸ vector genomes, at least about 10⁹, at least about 10¹⁰, and up to about 10¹⁵, up to about 10¹⁴, up to about 10¹², and may be from about 10⁸ to 10¹⁵, from about 10⁹ to about 10¹⁴, from about 10¹⁰ to about 10¹².

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (host) being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, MD 20857; (available at www(dot)fda(dot)gov/cder/guidance/index(dot)htm, the disclosure of which is incorporated herein by reference).

### Pharmaceutical Compositions

A pharmaceutical composition comprising a therapeutic vector, e.g. an AAV virus comprising a therapeutic vector, may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, or any other dosage form suitable for administration.

A therapeutic vector may be administered to the host using any convenient means capable of resulting in the desired reduction in disease condition or symptom. Thus, a therapeutic vector can be incorporated into a variety of formulations for therapeutic administration. More particularly, a therapeutic vector can be formulated into pharmaceutical compositions by combination with appropriate pharmaceutically acceptable carriers or diluents, and may be formulated into preparations in solid, semi-solid, liquid or gaseous form.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the infection to be treated. In some embodiments, formulations include a pharmaceutically acceptable carrier in addition to at least one active ingredient, such as a subject compound. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed compositions may comprise a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A therapeutic vector can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A therapeutic vector can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, or other route of administration, e.g., injection into the retina.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

In some instances, methods of treating a subject as described herein may include administering to the subject an effective amount of an agent that reduces RGC degeneration in the subject, as identified in a method of screening described herein.

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of treating a neurodegenerative condition in a subject, including by administering to the subject an effective amount of a therapeutic vector that reduces the prevalence of RGC degeneration. The subject kits may include any combination of components (e.g., reagents, cell lines, etc.) for performing the subject methods, such as e.g., methods of treating a neurodegenerative condition and/or methods of identifying a RAG target gene.

In some aspects, provided herein is a kit comprising an AAV vector, wherein the vector comprises a promoter that promotes expression of a RAG coding sequence specifically in RGCs, wherein the promoter is in operable linkage with an expression cassette; and instructions for use.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

### Single Cell Transcriptome Analysis of Regenerating RGCs Reveals Potent Glaucoma Neural Repair Genes

Axon regeneration holds great promise for neural repair of CNS axonopathies, including glaucoma. Pten deletion in retinal ganglion cell (RGC) promotes potent optic nerve (ON) regeneration after ON crush (ONC), but only a small population of Pten-null RGCs are actually regenerating RGCs (regRGCs); most surviving RGCs (surRGCs) remain non-regenerative. To identify regeneration-associated genes for neural repair of glaucoma, we developed a strategy to specifically label and purify regRGCs and surRGCs respectively from the same Pten deletion mice after ONC, in which they differ only in their regeneration capability. Smart-Seq2 single cell transcriptome analysis revealed novel regeneration-associated genes (Anxa2, Plin2, Mpp1, Acaa2, Spp1, Lgals1, Spc24) that significantly promote axon regeneration. The most potent of these, Anxa2, acts synergistically with its ligand tissue plasminogen activator in Pten deletion-induced ON regeneration. Anxa2 dramatically protects RGC somata and axons and preserves visual function in a clinically relevant model of glaucoma, demonstrating the exciting potential of this innovative strategy to identify novel effective neural repair candidates.

We previously explored RGC and ON neuroprotection in chronic optic neuropathy models but only used traumatic ONC model to study axon regeneration. Glaucoma differs from ONC because axon damage is incomplete, and therefore it is difficult to differentiate regenerating axons from surviving axons. However, the pro-regeneration genes identified from regenerating RGCs in the ONC model may be promising candidates for glaucoma neural repair, as showed by a recent study. Therefore, we first developed a tracing scheme to differentially label and isolate surviving RGCs with or without axon regeneration in Pten KO mice after ONC injury. We then compared the single cell transcriptomes of these RGCs by Smart-Seq2 to systematically cluster genes associated with axon regeneration, and identified seven novel pro-regeneration genes with various regeneration capacity. Finally, we demonstrated that overexpression of Anxa2 (Annexin A2) in RGCs achieved dramatic neuroprotection and visual function preservation in an experimental model of glaucoma. This proof-of-concept study demonstrates the feasibility and efficacy of this strategy in identifying the targetable downstream effectors of Pten. This strategy can also readily elucidate the downstream effectors of other single or combinatory regeneration approaches, and thus identify effective and safe neural repair targets for glaucoma and other CNS neurodegenerative diseases.

### Results

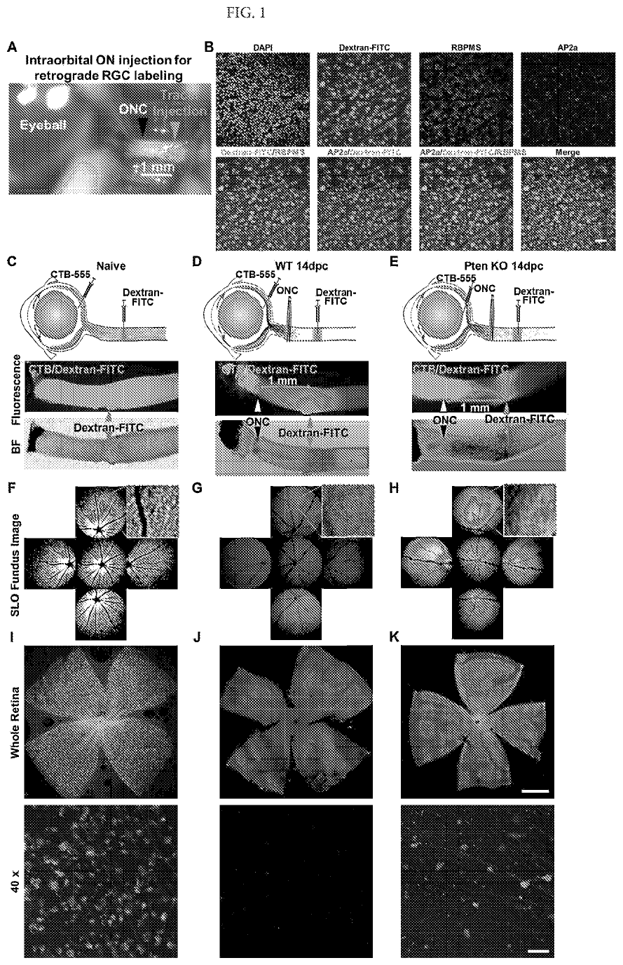

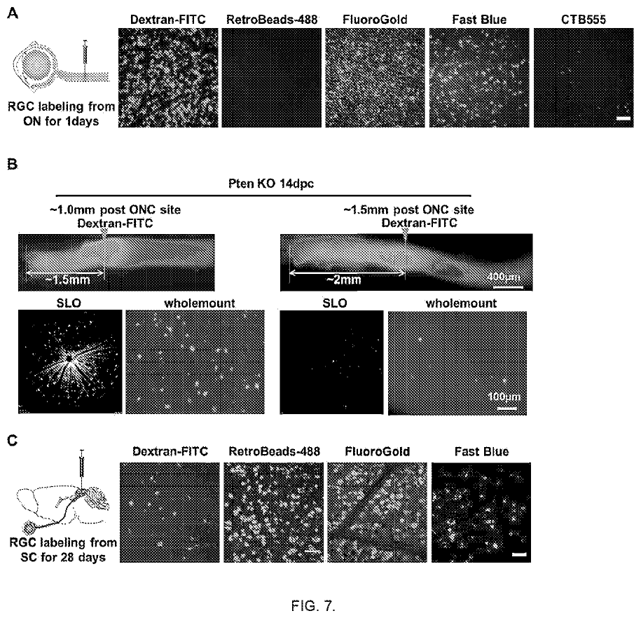

Strategies for labeling, isolating, and single cell transcriptome profiling of regenerating RGCs (regRGCs) and surviving RGCs without axon regeneration (surRGCs). To specifically label RGCs that send regenerating axons into ON distal to the crush site, we developed an intraorbital ON injection method to label RGC cell bodies by retrograde tracing of dye applied to regenerating axons. We exposed the intraorbital portion of ON about 2 mm distal to the eyeball by piercing the conjunctiva and tunneling beneath soft tissues and muscles from the lateral side of the mouse eye while avoiding injury to the retro-orbital sinus. Because the ONC site is usually ˜0.5 mm from the eyeball, we labeled regenerating axons by injecting neuronal tracer 1-1.5 mm distal to the crush site (FIG. 1A). We used dextran after testing several other tracers because of the superiority of its transport speed and retrograde labeling efficiency of RGCs after ON injection (FIG. 7A). In the naïve animal, intraorbital ON injection of dextran labeled more than 90% RBPMS⁺ RGCs but not neighboring AP2a⁺ amacrine cells (FIG. 1B). The entire retina was labeled uniformly, evidenced by homogenous RGC labeling detected by in vivo SLO retinal fundus imaging and wholemount retina histology (FIG. 1C,F,I). We further confirmed that this method did not label any RGCs in wildtype (WT) mice 14 days post ON crush (14dpc) in which there are no regenerating axons (FIG. 1D,G,J). In dramatic contrast, intraorbital ON injection of dextran labeled a distinct population of RGCs in Pten KO mice in which regenerating axons reach at least 1 mm beyond the crush site at 14dpc (FIG. 1E,H,K). These observations substantiate the reliability and efficiency with which this retrograde tracing method labels regenerating RGCs in vivo. We performed the injection at about 1 mm distal to the crush site (˜1.5 mm from the eyeball) because we found that much fewer regRGCs were labeled by an injection 0.5 mm more distal (˜2.0 mm from the eyeball), probably because far fewer regenerating axons elongated that distance at 14dpc in Pten KO mice (FIG. 7B). To pre-label the total population of RGCs before ONC to detect surviving RGCs (surRGCs) after crush, we used retrobeads as a neuronal tracer after superior colliculus (SC) injection because they retrogradely labeled RGCs more efficiently than other tracers that we tested and, more importantly, because of the superior stability of the signal, which persists in RGCs for at least several weeks (FIG. 7C).

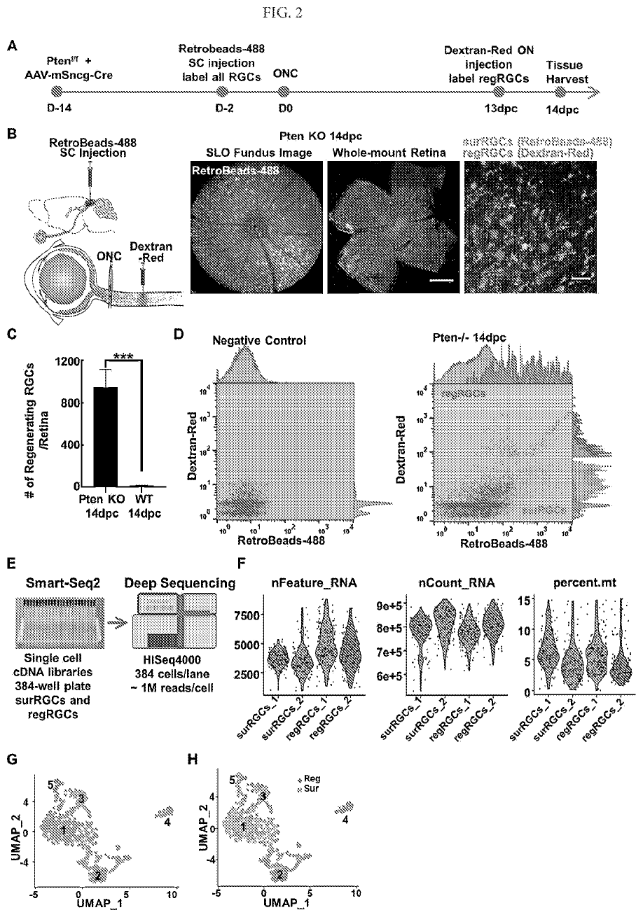

We used these retrograde tracing methods to finalize the time line of the labeling and the isolation procedures (FIG. 2A): 1) We induced Pten deletion in RGCs in Pten floxed mice by intravitreal injection of AAV-Cre driven by a mouse γ-synuclein promoter (mSncg), a RGC-specific promoter that we recently identified, 14 days before ONC; 2) We injected retrobeads-488 into the SC 2 days before ONC to label all RGCs with green fluorescence; 3) We performed intraorbital ON injection with dextran-Red to label regRGCs with red fluorescence 13 days post ONC (1 day before tissue collection at 14dpc). This strategy detected around 900-1000 regenerating RGCs/retina in Pten KO mice at 14dpc (FIG. 2B,C), similar to published regenerating axon counting. 4) We used FACS sorting to isolate separately dextran-Red positive regRGCs and retrobeads-Green positive but red negative surRGCs (FIG. 2D). These cells were individually distributed into each well of a 96-well plate and later transferred to a 384-well plate to generate a single cell Smart-Seq2 cDNA library according to an established protocol. 384 cells with distinct sequence barcodes were sequenced together in one HiSeq lane to acquire roughly 1 million sequencing reads/cell and repeated once with an independent group of mice (FIG. 2E). Based on the quality control thresholds of more than 900 genes/cell, 0.5 million reads/cell, and fewer than 15% mitochondria genes (FIG. 2F), we acquired 340 regRGCs and 290 surRGCs that had at least one of the 7 pan-RGC markers (RBPMS, Thy1, Slc17a6/vGLUT2, Pou4f1-3/Brn3a-c) and very low or no Pten expression.

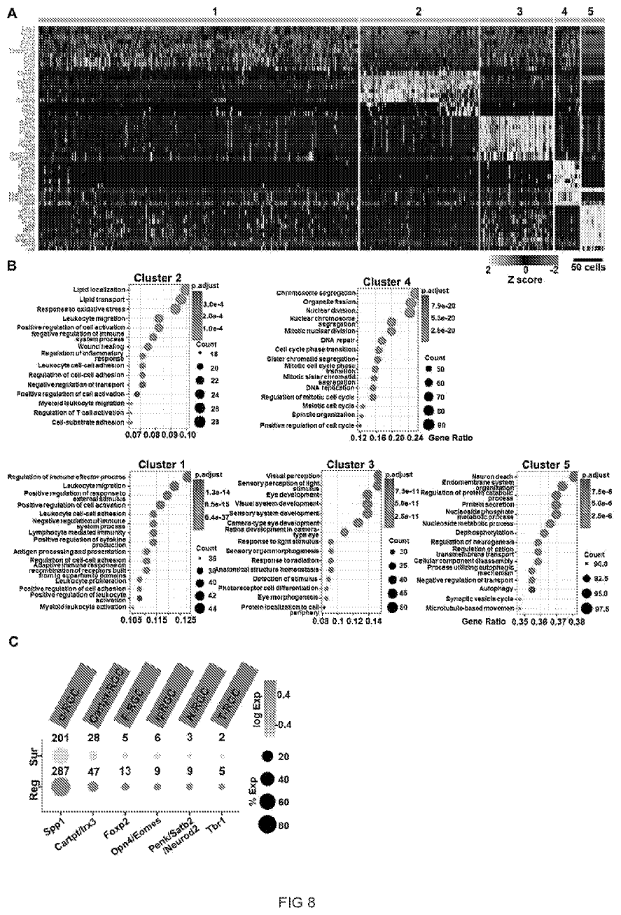

We pooled the Smart-Seq2 transcriptomes of a total of 630 regRGCs and surRGCs and clustered them into 5 molecular clusters (FIG. 2G). Superimposition of regeneration identities demonstrated that cluster 2 and cluster 4 were enriched with regRGCs (FIG. 2H), suggesting that regRGCs and surRGCs tend to segregate into distinct molecular clusters. Distinct gene sets were associated with each cluster (FIG. 8A, Table S1). Further gene ontology (GO) enrichment analysis showed distinct biological pathways associated with each cluster (FIG. 8B): 1) regRGC-enriched cluster 2 and cluster 4 were associated with lipid transport, cell migration, cell adhesion, and wound healing, or cell mitosis, cell cycle, and DNA replication and repair, respectively; 2) Cluster 1 were associated with immune/inflammation responses; cluster 3 genes were associated with visual function and development; and cluster 5 were associated with neuron death, and synapse structure and function. Using the extensive mouse RGC atlas generated by droplet-based scRNA-seq as a reference, we confirmed that the majority of Pten KO surRGCs and regRGCs were Spp1⁺α-RGCs (FIG. 8C), consistent with a previous report that α-RGC is the general RGC subtype for survival and regeneration after Pten KO in the ONC model. Other major RGC subtypes' marker genes were also detected in our samples, including Cartpt-RGCs, F-RGCs (Foxp2+), ipRGCs (Opn4/Eomes+), N-RGCs (Neurod2/Penk/Satb2+), and T-RGCs (Tbr1+), although most of the marker genes were higher expressed in regRGCs.

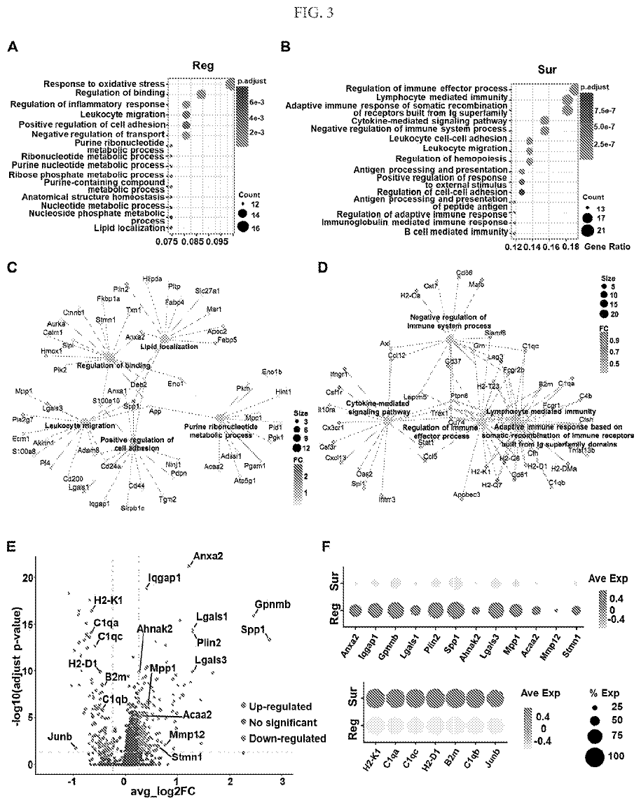

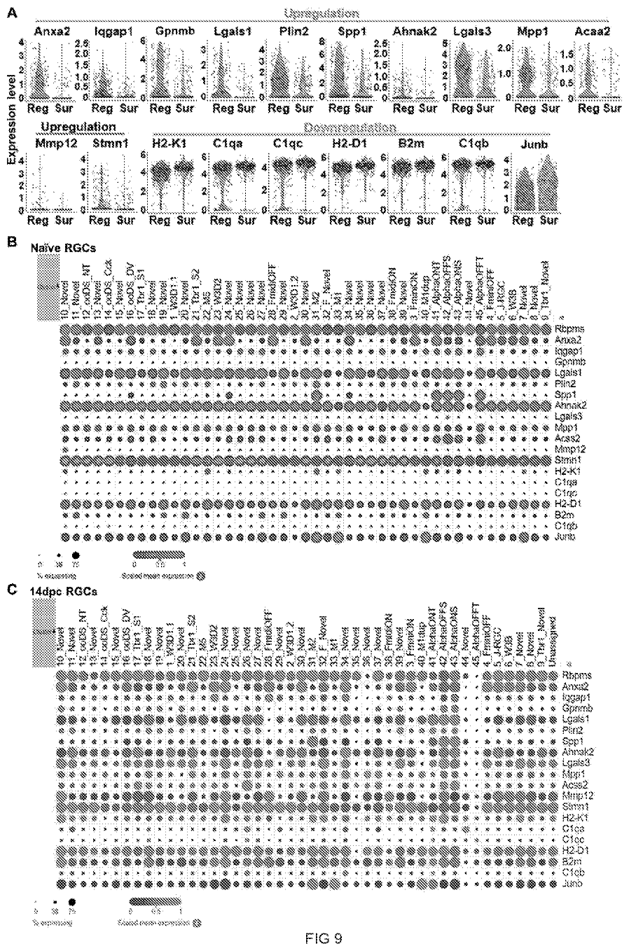

Regeneration-associated genes revealed by comparison of Smart-Seq2 transcriptomes of regRGCs and surRGCs. To identify genes that are associated with axon regeneration, we directly compared the Smart-Seq2 transcriptomes of regRGCs and surRGCs because the only difference between these two populations is whether or not their axons regenerate. This pseudo-bulk comparison revealed 168 upregulated genes (Table S2) and 116 downregulated genes in regRGCs as differentially expressed genes (DEGs) (Table S3). Some of the upregulated DEGs are known to be involved in axon regeneration and cell survival, such as Spp1, Lgals3, Mmp12, and Stmn1. GO analysis of upregulated DEGs in regRGCs showed enrichment of biological pathways related to regulation of lipid localization, cell adhesion, migration, and metabolic processes (FIG. 3A,C). Based on this association, we marked some of the genes (most significant and enriched in these GO pathways) in the volcano figures and showed their expression in regRGCs and surRGCs, including Anxa2, Iqgap1, Gpnmb, Lgals1, Lgals3, Plin2, Spp1, Mpp1, Acaa2, and Ahnak2 (FIG. 3E,F). The downregulated genes were associated with immune responses (FIG. 3B,D-F), such as H2-K1, H2-D1, B2m, and C1q family; we did not pursue them further in this study. We confirmed the differential expression of these genes in regRGCs and surRGCs (FIG. 9A) and their expression in different RGC subtypes by searching the online scRNA-seq database of adult mouse RGCs (FIG. 9B,C).

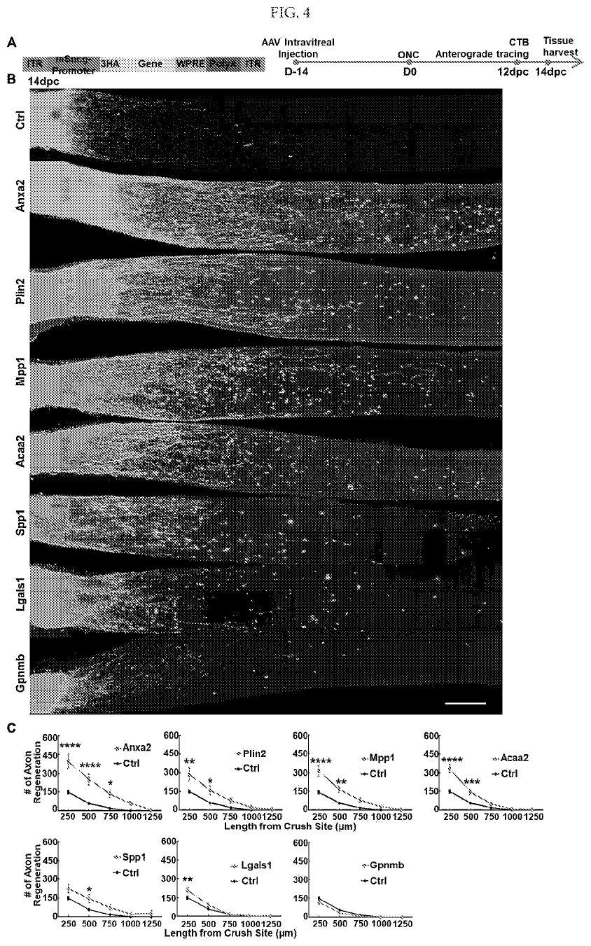

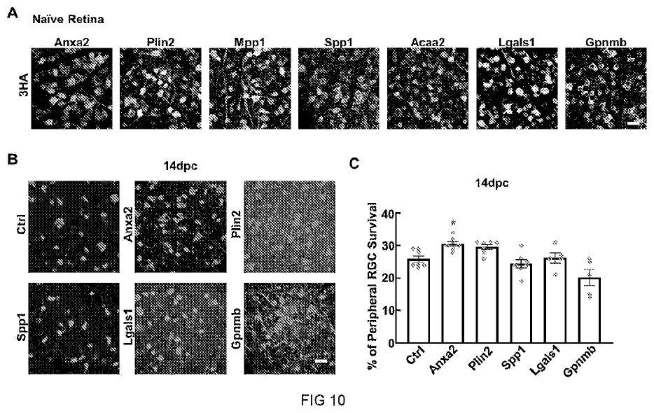

Six of seven tested DEGs promote significant axon regeneration after ONC injury and Anxa2 is the most potent pro-regeneration gene. We successfully generated AAVs to express seven regRGCs-upregulated DEGs driven by the mSncg promoter in RGCs specifically and tested them in axon regeneration after ONC injury (FIG. 4A, FIG. 10A). Because the coding sequences of Iqgap1 and Ahnak2 are too large to fit into AAV vectors, we did not test them. Six out of seven DEGs, Anxa2, Plin2, Mpp1, Acaa2, Spp1, and Lgals1 but not Gpnmb, promoted significant axon regeneration, but to a differing extent (FIG. 4B,C). Anxa2 was the most potent pro-regeneration gene and the only one to increase RGC survival (FIG. 10B,C). Therefore, we next focused on characterizing Anxa2.

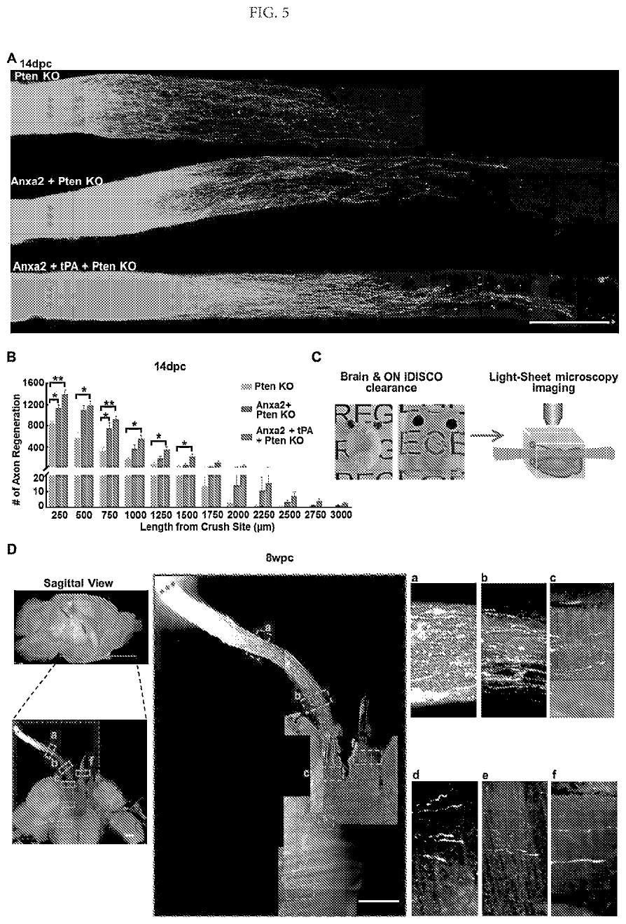

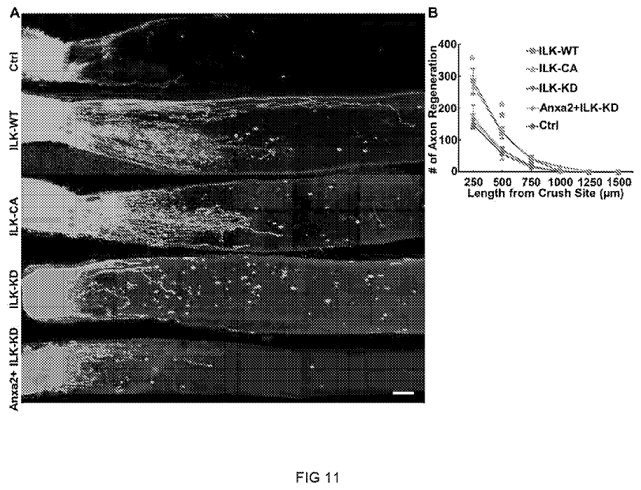

Anxa2 and its cell surface ligand tissue plasminogen activator (tPA) act synergistically with Pten deletion to promote potent axon regeneration. We found that RGC-specific Anxa2 overexpression and Pten deletion promoted more axon regeneration than Pten KO alone at 14dpc (FIG. 5A,B), suggesting that increasing Anxa2 and inhibiting Pten would act synergistically to enhance axon regeneration. Anxa2 functions as a cell surface co-receptor for tissue plasminogen activator (tPA) and is therefore involved in plasmin-related cellular processes, such as fibrinolysis, extracellular matrix (ECM) degradation, cell migration and cancer cell invasion. Adding tPA further increased axon regeneration (FIG. 5A,B), suggesting a role for plasmin-related functions, such as ECM degradation, in axon regeneration. At 8-week post crush (8wpc), we took light-sheet images of optical cleared brain with both ONs attached (FIG. 5C). Triple treatment consisting of Anxa2 overexpression, Pten KO, and tPA administration induced regenerating axons to grow through optic chiasm into optic tract, and some to cross to the contralateral side and grow into contralateral ON (FIG. 5D). RGC survival was unchanged by overexpressing Anxa2 in Pten KO mice but significantly increased after adding tPA (FIG. 11), further confirmation of tPA's beneficial effects. Although current understanding is that neuron survival is not invariably linked proportionately with axon regeneration, we cannot exclude the possibility that tPA increased axon regeneration through increasing RGC survival.

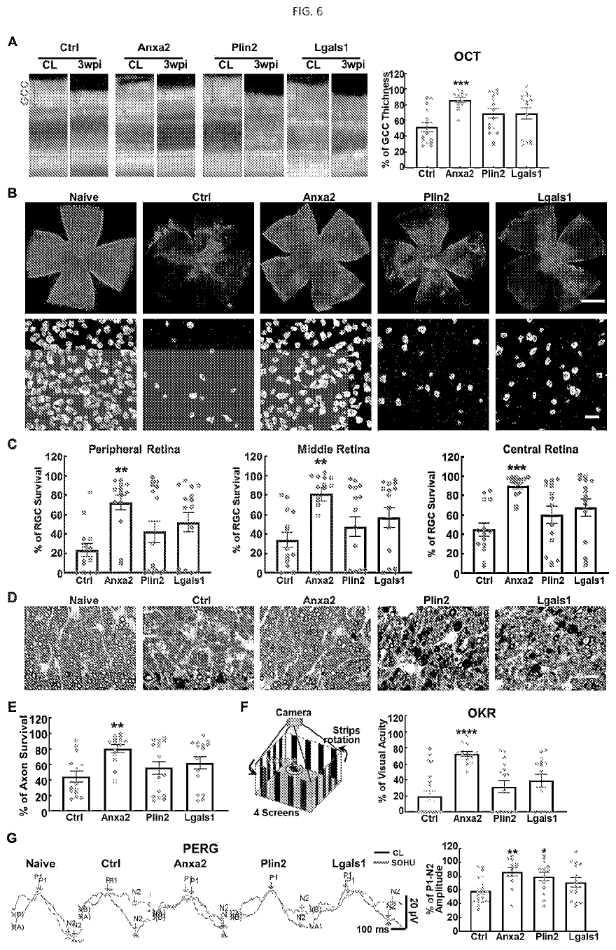

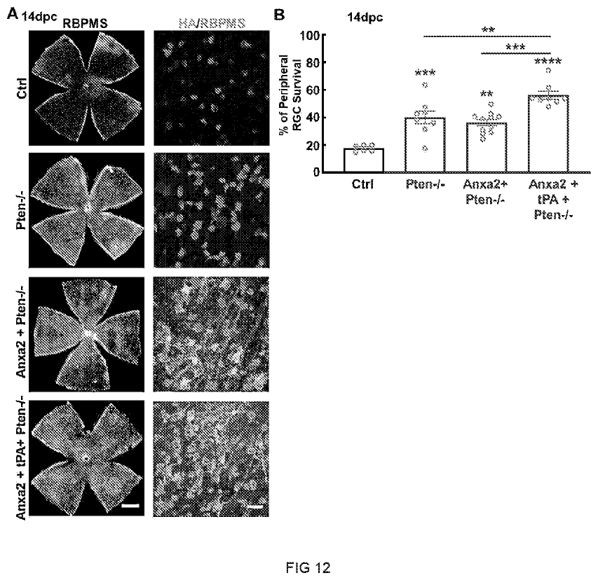

Anxa2 overexpression significantly promotes survival of RGC soma and axon, and preserves visual functions in the SOHU glaucoma model. Lastly, we asked whether the pro-regeneration genes that we identified through the traumatic ON injury model would enhance neural repair effect of RGC/ON and visual function in the more common, chronic optic neuropathy, glaucoma. We took advantage of our recently developed clinically relevant silicone oil-induced ocular hypertension (SOHU) mouse glaucoma models⁵⁷⁻⁵⁹ to test whether overexpression of Anxa2, or another two weaker pro-regeneration genes Plin2 and Lgals1, would benefit glaucomatous neurodegeneration. We injected AAV-mSncg-transgene into the vitreous to infect RGCs 14 days before inducing SOHU glaucoma to allow the transgene expression. We then performed intracameral injection of SO into one eye to mimic the secondary glaucoma found in patients when SO induces elevated IOP by pupillary blocking and injected PBS into the contralateral eye as an internal control (FIG. 12A,B). Thinning of the retinal nerve fiber layer (RNFL) measured by optical coherence tomography (OCT) serves as a critical biomarker for optic neuropathies and related neurodegeneration in the clinic. We and others use OCT to measure the thickness of the retinal ganglion cell complex (GCC) in living mice, including RNFL, ganglion cell layer (GCL) and inner plexiform layer (IPL), to serve as an indicator of RGC/ON degeneration in diverse optic neuropathy mouse models. In vivo OCT imaging showed significant thinning of the GCC in SOHU eyes compared to contralateral control eyes in control group animals injected with control AAVs, at 3-week post SO injection (3wpi) (FIG. 6A). GCC thinning was concurrent with significant IOP elevation (FIG. 12C). Thus we were able to replicate the SOHU glaucoma model, with stable IOP elevation and severe glaucomatous neurodegeneration, which faithfully simulates human pupillary blocking glaucoma caused by surgical use of SO. Only Anxa2, but not Plin2 or Lgals1, significantly increased GCC thickness in SOHU eyes (FIG. 6A). Histological analysis of post mortem retina wholemounts consistently showed that treatment with Anxa2 strikingly increased RGC survival throughout the whole retina (FIG. 6B,C). In contrast, neither Plin2 nor Lgals1 showed a significant effect. Quantification of surviving axons in semi-thin sections of ONs further confirmed that overexpression of Anxa2, but not of Plin2 or Lgals1, achieved dramatic axon protection in glaucoma (FIG. 6D,E).

In addition to morphological protection, preservation of visual function is of utmost importance for neural repair. We next investigated whether RGC-specific expression of pro-regeneration genes preserved visual function in glaucomatous mice. Optokinetic tracking response (OKR) is a natural reflex that objectively assesses mouse visual acuity. The mouse eye will only track a grating stimulus that is moving from the temporal to nasal visual field, which allows both eyes to be measured independently. Consistent with our post-mortem histological and in vivo morphological results, Anxa2 significantly preserved visual acuity of the glaucomatous eyes (FIG. 6F). The pattern electroretinogram (PERG), a sensitive electrophysiological assay of general RGC function, is obtained in response to a visual stimulus consisting of contrast reversal patterned gratings at constant mean luminance. Because our PERG system can measure both eyes at the same time, there is an internal control to serve as a reference and normalization to minimize variations. We previously employed PERG successfully to examine the changes of RGC function in the SOHU glaucoma model. The peak-to-trough (P1-N2) amplitude ratio of the SOHU eyes to contralateral (CL) eyes increased significantly after Anxa2 and Plin2 overexpression in RGCs (FIG. 6G). Taken together, these results show convincingly that Anxa2 overexpression in RGCs achieves significant neural repair in the SOHU glaucoma model, demonstrated by striking neuroprotection of RGCs and ONs, and preservation of visual functions.

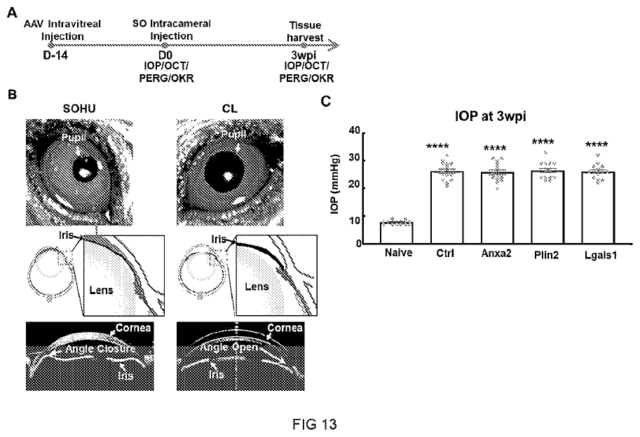

Cluster 4 regRGCs are highly enriched with cell cycle S/G2-M phase-related genes but do not proliferate; Spc24, one of the mitotic genes, induces significant axon regeneration after ONC. Because of the uniqueness of the cluster 4 RGCs that are enriched with well-known genes associated with S/G2-M phases of mitosis (FIG. 13A,B), we tested the effects of some of the top mitotic genes. We first, however, studied whether Pten deletion would alter RGC proliferation. Adult neurons are terminally differentiated post-mitotic cells due to their arrested cell cycle. Because Pten is a tumor suppressor gene, its deletion might cause these cells to re-enter the cell cycle. We performed an EdU pulse chase experiment in mouse retinas with or without Pten deletion, and with or without ONC. We failed to detect any RBPMS⁺ RGCs colocalized with EdU⁺ cells in WT or Pten KO retinas without ONC injury, indicating no proliferating RGCs in adult mouse with or without Pten deletion (FIG. 13C). Most importantly, we found no regRGCs labeled by dextran retrograde ON tracing that were EdU positive (FIG. 13C), indicating that regRGCs also did not actively proliferate. Overexpression of Spc24 in RGCs induced significant axon regeneration (FIG. 13D,E) but had no effect on RGC survival (FIG. 13F,G). None of other S/G2-M phase associated genes that we tested promoted significant axon regeneration (FIG. S8). In summary, the small cluster of regRGCs enriched with mitotic S/G2-M phase genes were not in an active proliferation stage, and one of the DEGs of this cluster, Spc24, enhanced axon regeneration.

We developed a novel intraorbital ON retrograde tracing technique, which enabled us for the first time to purify regenerating and non-regenerating RGCs from the same animals with the same genetic background/modification/injuries. These RGCs differ only in their capacity for axon regeneration. Profiling and comparing the transcriptomes of regRGCs and surRGCs at single cell level allowed us to unbiasedly determine genes associated with axon regeneration. Further characterization of the effects of some of these genes on axon regeneration in the ONC model and neuroprotection in the glaucoma model demonstrated the power and efficiency of this comprehensive but very focused strategy. We obtained the following findings: 1) The unique biological pathways associated with distinct clusters of RGCs are readily determined at the single cell molecular level; 2) Direct comparison of regRGCs and surRGCs directly identifies a list of regeneration-associated genes, many of which we showed to be pro-regeneration molecules; 3) The most potent pro-regeneration molecule, Anxa2, achieves the most striking neuroprotection in the glaucoma model. This strategy can be used to identify downstream effectors or regulatory networks of other single or combinatory genes that promote significant axon regeneration, which will rapidly and significantly advance the axon regeneration field. The common effectors the multiple signaling pathways involved in axon regeneration and neuronal survival will be promising targets for developing effective and safe neural repair therapies for neurodegenerative diseases. Additionally, the common regeneration-associated genes that are not present in non-regenerating RGCs provide potential regeneration markers that are also desperately needed in the field.

Membrane structure/signaling molecules are critical axon regeneration. Anxa2 is the most significant regRGCs-enriched gene and promotes the most potent axon regeneration among the genes we tested. It is a Ca²⁺-dependent phospholipid and F-actin-binding membrane protein with diverse roles in cytoskeletal-membrane dynamics, exocytosis, endocytosis, cell polarity, and lipid raft signaling. Anxa2 is critical for Ca²⁺-dependent exocytosis of hormones in neuroendocrine cells, indicating its roles in regulating neuronal activity, growth, and plasticity through secretion of neurotransmitters and neuropeptides. The most studied function of Anxa2 is related to the co-receptor complex that it forms with S100A10 on the cell surface. This complex attracts tPA locally to generate plasmin and therefore to promote plasmin-mediated ECM degradation. Interestingly, LRP1 is another tPA receptor that is also involved in axon regeneration. Our findings that Anxa2 promotes axon regeneration and that tPA further increases Anxa2/Pten modulation-induced axon regeneration, raise the possibility that the Anxa2-tPA complex on the surface of plasma membrane might serve as a proteolytic center that generates plasmin and therefore clears ECM nearby to make room for axons to grow. This action could facilitate regenerating axon elongation, a mechanism that has been confirmed for Anxa2-tPA-mediated cell adhesion, migration, and cancer cell invasion. Anxa2/S100A10 also interacts with Ahnak to regulate Ca²⁺-dependent exocytosis, L-type voltage-gated calcium channels, synaptic transmission, and membrane repair. Interestingly, both S100A10 and Ahnak are also in our regRGC-enriched gene list, further supporting the importance of the Anxa2/S100A10/Ahnak/tPA axis in axon regeneration.

Three other pro-regeneration genes identified by this study, Mpp1 (Membrane Palmitoylated Protein 1), Spp1 (Secreted Phosphoprotein 1, osteopontin) and Lgals1 (Galectin-1), share many similarities with Anxa2. Mpp1 is also a plasma membrane protein that form diverse protein complexes and is involved in cell structure, polarity, cell adhesion and migration, synaptogenesis, raft formation, and signal transduction. Mpp1 is required for insulin-stimulated activation of H-Ras, a growth factor-initiated signaling pathway that is known to promote pro-axon regeneration. Spp1 interacts with multiple integrins and is involved in cytokine secretion, cellular differentiation, adhesion, migration, and would healing. Galectin 1 (Lgals1), which has known roles in axon growth, axon guidance, and axon regeneration, belongs to a family of glycan-binding proteins that recognize distinct sets of glycosylated proteins or lipids at the cellular surface or ECM. Lgals1 also interacts with β1 integrin to regulate neural progenitor cells. Anxa2 also interacts with integrins and activates integrin linked kinase (ILK), which is a vital signaling protein that mediates integrin regulation of cell adhesion, proliferation, migration, angiogenesis, and actin-cytoskeleton dynamics. ILK activity is inhibited by Pten. Thus, Anxa2, Spp1, and Lgals1, acting as the downstream effectors of Pten inhibition, may promote axon regeneration by activating the integrin-ILK pathway. More broadly, we hypothesize that cell membrane structure molecules that are involved in cell adhesion/migration/invasion/exocytosis are important for axon regeneration, possibly through mediating interaction of intracellular and extracellular signaling and reorganizing local cytoskeleton/ECM. Additional findings that cell surface molecules neural cadherin and thrombospondin 1, complement proteins in the ON locally, cytoskeleton stabilization and motor molecules, promote axon regeneration further support this theme. Another pro-regeneration gene we found, Plin2, is the coat protein of lipid droplets and important for cellular lipid homeostasis. Consistently, Lipin 1, an enzyme that inhibits membrane phospholipid, also inhibits axon regeneration. Detail profile and characterization of molecules in the growth cones of regenerating axons may shed more light on the molecular mechanisms of local membrane molecules in axon regrowth.

From axon regeneration to neural repair. The striking neuroprotection produced by Anxa2 in the glaucoma model is a surprising finding, because it only marginally increases RGC survival after ONC injury. However, although ONC is extensively used as a convenient model of optic neuropathy and often as a surrogate glaucoma model, RGC/ON may respond very differently to the same neural repair treatments depending on whether they are injured by traumatic injury or IOP elevation. One previous study also found that pro-regeneration genetic modulation significantly promotes neural repair in glaucoma model. The wide range of cellular functions and signaling cascades mediated by Anxa2 cited above may contribute to the significant neuroprotection and visual function preservation in the SOHU glaucoma model, but further investigations will be necessary to understand the precise mechanisms. The pro-regeneration activity of Anxa2 may also be critical for the neuroprotection in glaucoma, although it is difficult to appreciate how much axon regeneration is indeed induced by Anxa2 in this disease model in which axon damage is incomplete. The weaker pro-regeneration genes Plin2 and Lgals1 barely increase neuronal survival and visual function in the glaucoma model, which is additional evidence that regeneration capability may be linked to neuroprotection potential. The present results represent an early, but very encouraging and compelling confirmation of this search strategy: identifying pro-regeneration genes associated with regRGCs in the ONC model as promising candidates for neural repair in glaucoma.

Pten deletion on cell cycle re-entering of adult neuron. Our detection of a distinct group of regRGCs enriched with S/G2-M phases-associated genes is also surprising. It is conceivable that Pten deletion may remove the inhibition of mitosis in these adult neurons and allow them to re-enter the cell cycle, although our EdU labeling experiment did not indicate that regRGCs actively proliferate. However, the possibility remains that the Pten-deleted regRGCs re-enter into cell cycle and that the time point that we checked (two weeks after ONC) is not long enough to detect the incorporation of EdU for DNA replication. Alternatively, the upregulation of S/G2-M phases-associated genes may only prime the mature neurons for re-growth but not push them into actual cell division. One of the cell cycle-related genes that we tested, Spc24, can promote significant axon regeneration, suggesting that these genes may have additional functions that do not necessarily drive the cells into proliferation. Further studies using our strategy to explore regenerating RGCs after modulating other genes, exploring longer and additional time points, and investigating other cell cycle-related genes will be helpful to better understanding these findings. The present study may not entirely solve concerns over the safety of Pten modulation in neural repair, but it is reassuring to know that many regRGCs do not upregulate mitotic genes after Pten deletion, and that the regRGCs enriched with these genes are not actively proliferating.

In summary, we present a powerful and efficient tracing methodology to distinctly label and isolate regRGCs and surRGCs from the same animals after the same genetic modulation, injury and treatment, which allows us to definitively compare single cell transcriptomes to identify truly regeneration-associated genes. Four out of seven pro-regeneration genes we identified through this study, Anxa2, Mpp1, Spp1, and Lgals1, share many similarities, such as involvement in plasma membrane-binding, local membrane-cytoskeleton dynamic/lipid rafts formation and integrin signaling, and exocytosis. Based on these results, together with the synergistic effect of tPA with Anxa2 and Pten, we hypothesize that plasma membrane-ECM remodeling and signaling cascades play critical roles in axon regeneration and regrowth. The differentiation strategy that we employed detects without bias distinct RGC clusters that are preferentially accumulated with regRGCs or surRGCs and have signature genes and biological pathway profiles. For example, a smaller portion of adult neurons becoming “mitotic-like” cells after Pten deletion are enriched with multiple S/G2-M phases-genes but do not proliferate, whereas more Pten-null RGCs remain post-mitotic. Finally, and extremely importantly, we found that Anxa2 is a very promising neuroprotectant due to its dramatic protection of glaucomatous RGCs/ONs and visual function deficits, which validates the strategy of searching for neural repair candidates among the potent pro-regeneration molecules. This whole strategy is likely to be applicable to other neurodegenerative diseases or trauma associated with long axons, such as motor neuron degenerative diseases and spinal cord injury.

### Materials and Methods

| Reagent | | | | |
|---|---|---|---|---|
| type | | | | |
| (species) or | | Source or | | |
| resource | Designation | reference | Identifiers | Dilution |
| strain, | C57BL/6J | Jackson | 000664 | |
| strain | | Laboratories | | |
| background | | | | |
| (Mus | | | | |
| musculus) | | | | |
| strain, | | | | |
| strain | B6.129S4- | | | |
| Pten^{tm1Hwu}/J | Jackson | | | |
| Laboratories | 006440 | | | |
| background | | | | |
| (Mus | | | | |
| musculus) | | | | |
| antibody | Anti-RBPMS (guinea | Custom-made by | | 1:4000 |
| | pig polyclonal) | ProSci | | |
| antibody | Anti-AP2α (mouse) | Developmental | 3B5 | 1:100 |
| | | Studies Hybridoma | | |
| | | Bank | | |
| antibody | Anti-3HA (rat) | Roche | 11867423001 | 1:200 |
| antibody | Alexa Fluor 647 | Jackson | 106-605-003 | 1:200 |
| | Goat anti-Guinea Pig | ImmunoResearch | | |
| | IgG | | | |
| antibody | Cy3 Goat anti-rat | Jackson | 112-165-167 | 1:200 |
| | IgG | ImmunoResearch | | |
| antibody | Cy2 Goat anti- | Jackson | 115-225-166 | 1:200 |
| | mouse IgG | ImmunoResearch | | |
| Tracing dye | Cholera Toxin | ThermoFisher | C22843 | |
| | Subunit B | scientific | | |
| | (Recombinant), | | | |
| | Alexa Fluor ™ 555 | | | |
| | Conjugate | | | |
| racing dye | Dextran-FITC, 3000 | ThermoFisher | D3306 | 3% w/v |
| | MW, anionic, lysine | Scientific | | in PBS |
| | fixable | | | |
| Tracing dye | Dextran, Texas Red, | | | |
| | 3000 MW, | | | |
| lysine Fixable | ThermoFisher | D3328 | 3% w/v | |
| | Scientific | | in PBS | |
| Tracing dye | FluoroGold | ThermoFisher | NC1363013 | 2.5% |
| | | Scientific | | |
| w/v in PBS | | | | |
| Tracing dye | Dil Cell-Labeling | ThermoFisher | V22885 | 1 mM |
| | Solution | Scientific | | solution |
| Tracing dye | FastBlue | PolySciences | 17740-1 | 5% w/v |
| | | | | in PBS |
| Tracing dye | RetroBeads Green | | | |
| (488) Solution | Lumafluor | | | |
| Protease | | | | |
| The tissue | Sigma-Aldrich | 612200-M | 30 U/uL | |
| plasminogen | | | | |
| activator (human) | | | | |
| chemical | Silicone oil | Alcon Laboratories | 1,000 mPa · s, | |
| Silikon | | | | |
| chemical | AMES' Medium | Sigma-Aldrich | A1420-10x1L | |
| chemical | Papain | Worthington- Fisher | LS003126 | |
| chemical | Ovomucoid | Worthington- Fisher | LS003087 | |
| chemical | L-cysteine | Sigma-Aldrich | C1276-10g | |
| chemical | Methanol | Fisher Scientific | A452-4 | |
| chemical | Dichloromethane | Sigma-Aldrich | 34856-1L | |
| chemical | Dibenzyl ether | Sigma-Aldrich | 33630-1L | |
| chemical | | | | |
| software, algorithm | GraphPad Prism7 | GraphPad Software | | |
| software, algorithm | Fiji/ImageJ | NIH | | |
| software, algorithm | R for statistical | | | |
| | computing version | | | |
| | 4.0.2 | | | |
| software, algorithm | StringTie v2.1.7 | Pertea et al., 2016¹⁰⁸ | | |
| software, algorithm | Hisat2 v2.2.1 | Kim et al., 2019¹⁰⁹ | | |
| Primers | Primer for | IDT | Table S4 | |
| | overexpression | | | |
| | cloning | | | |
| device, equipment | LSM 880 confocal | Zeiss | LSM 880 | |
| | microscopy | | | |
| device, equipment | Keyence | Keyence | BZ-X800 | |
| | fluorescence | | | |
| | microscopy | | | |
| device, equipment | Zeiss M2 | Zeiss | | |
| | fluorescence | | | |
| | microscopy | | | |
| device, equipment | LaVision, Light | Miltenyi Biotec | | |
| | Sheet Ultramicroscope | | | |
| | II | | | |
| device, equipment | BD Influx System | BD Biosciences | | |
| | Cell Sorter | | | |
| device, equipment | Illumina HiSeq 4000 | Illumina | | |
| | sequencer | | | |
| device, equipment | | | | |
| Heidelberg SLO & | | | | |
| OCT | | | | |
| Heidelberg | | | | |
| device, equipment | | | | |
| Pattern | | | | |
| electroretinogram | | | | |
| (PERG) | | | | |
| Intelligent Hearing | | | | |
| Systems | | | | |
| device, equipment | | | | |
| Opto-kinetic | | | | |
| response (OKR) | | | | |

Mice. C57BL/6J WT and B6.129S4-Pten^{tm1Hwu}/J mutation (Pten^{(f/f)} male and female mice were purchased from Jackson Laboratories (Bar Harbor, Maine). All mice were housed in standard cages on a 12-hour light-dark cycle. All experimental procedures were performed in compliance with animal protocols approved by the IACUC at Stanford University School of Medicine.

Constructs and AAV production. The coding regions of Anxa2, Plin2, Mpp1, Acaa2, Spp1, Lgals1, Gpnmb, Spc24, Ube2c, Birc5, Cdc20, Pcna, Tpx2, and Hmgb2 were amplified from different mouse tissue cDNA with Q5 high-fidelity DNA Polymerase (NEB, M0491L) and cloned into the pAM-AAV-mSncg-3HA-WPRE or pAM-AAV-CS265-3HA-WPRE backbone with EcoRI, MluI, XhoI, or HindIII sites. The pAM-AAV-mSncg-Cre-WPRE plasmid was published previously. The maxi-precipitation of the constructs was performed by following the manual of Endo-Free Plasmid Maxi kit (Omega Bio-tek, D6926-03/101319-342). The detailed procedure of the AAV production has been described previously. Briefly, AAV plasmids containing the target genes were co-transfected with pAAV2 (pACG2)-RC triple mutant (Y444, 500, 730F) and the pHelper plasmid (StrateGene) into HEK293T cells by the PolyJet (SignaGen Laboratories, SL100688) transfection reagent. After transfection for 72 hours, the cells were lysed and purified by two rounds of cesium chloride density gradient centrifugation. The AAV titers of target genes were determined by real-time PCR and diluted to 1.5×10¹¹ vector genome (vg)/ml for intravitreal injection, respectively.

Intravitreal injection. Mice were anesthetized by xylazine and ketamine based on their body weight (0.01 mg xylazine/g+0.08 mg ketamine/g). For AAV intravitreal injection, a pulled and polished microcapillary tube was inserted into the peripheral retina of around 4-week-old mice just behind the ora serrata. Approximately 2 μl of the vitreous was removed to allow injection of 2 μl AAV into the vitreous chamber. The mice were housed for an additional 2 weeks after AAV injection to achieve stable target genes expression. For anterograde tracing of regenerating axons, 2 μl of 2 μg/μl cholera toxin subunit B-Alexa 555 (CTB555, Invitrogen) was used for intravitreal injection. For the combinatory treatment of Anxa2 overexpression, tissue plasminogen activator (tPA, Sigma, 612200-m) and Pten KO, an AAV mixture with equal amount of Anxa2 and Cre was intravitreally injected into the Pten floxed mouse eyes 14 days before ONC, and 2 μl of 30 U/μl tPA was intravitreally injected twice/week after ONC.

Optic nerve crush (ONC). ONC was performed 2 weeks following AAV injection when mice were about 7-8 weeks of age. The ON was exposed intraorbitally at the 12 o'clock position while care was taken not to damage the underlying retro-orbital sinus, and crushed with a jeweler's forceps (Dumont #5; Fine Science Tools, Foster City, California) for 5 seconds approximately 0.5 mm behind the eyeball. Eye ointment containing neomycin (Akorn, Somerset, New Jersey) was applied to protect the cornea after surgery.

Retrograde labeling of regenerating RGCs by intraorbital ON injection. Pten KO mice were anesthetized by xylazine and ketamine based on their body weight (0.01 mg xylazine/g+0.08 mg ketamine/g). The ON was exposed intraorbitally ˜1.5-2 mm distal to the eyeball by lateral canthotomy through the conjunctiva and beneath soft tissues and muscles at the 9 o'clock position without injuring the retro-orbital sinus. The ON was immobilized by placing a piece of tissue paper between the ON trunk and surrounding soft tissue and the dura pierced by a 33g needle at the injection site, ˜1 mm distal to ONC site. A glass micropipette connected to a 50 μL microsyringe (80900, Hamilton) attached to a Micro4 controller was used to deliver ˜60 nL dextran into the ON through the pre-made hole, at a speed of 100 nL/min. Dye leaking at the injection site was removed by the tissue paper. After the injection, ointment containing neomycin was applied to protect the cornea, and mice were placed on heating pad for recovery. Mice were housed for 24 hours before tissue collection.

Retrograde labeling naive RGCs by superior colliculus (SC) injection. The detailed procedure of the micro-injection into the SC has been described previously. In brief, the adult mice were anesthetized by xylazine and ketamine based on their body weight (0.01 mg xylazine/g+0.08 mg ketamine/g) and fixed on a mouse adaptor attached to a digital stereotaxic instrument (68025, RWD Life Science). The bregma was set as the origin of anterior to posterior (AP), medial to lateral (ML) and dorsal to ventral (DV), and the same ML and DV of the lambda was aligned to the bregma. The horizontal plane of the mouse skull was calibrated by adjusting the left hemisphere point (AP:−2.00, ML:2.50) to the same DV as the contralateral point (AP:−2.00, ML:−2.50). The SC coordinates for 4 sites and 3 depths were located and drilled: AP:−3.55, ML:0.6, DV−1.25/−1.60/−2.00, AP:−3.55, ML:−0.6, DV−1.25/−1.60/−2.00, AP:−3.92, ML:0.8, DV−1.25/−1.50/−1.75 and AP:−3.92, ML:−0.8, DV−1.25/−1.50/−1.75. A pulled-glass micropipette fused to a 10 μL syringe (80314, Hamilton) filled with mineral oil was controlled by micro syringe pump (Micro4™, World Precision Instruments, LLC) at the speed of 250 nL/min for 1 minute per site. About 4 μL Dextran-FITC, Retrobeads-488, FluoroGold, or Fast Blue were injected into both SCs.

Retina cell dissociation and FACS purification. We followed the procedure described in a previous publication. Briefly, mouse retinas were dissected out in AMES solution (saturated with 95% O2/5% CO2) and then digested in papain (Worthington, LS003126) with L-cysteine (Sigma, C1276-10g) for 10-15 minutes at 37°. The single cell suspension was achieved by manual trituration in ovomucoid solution (Worthington, LS003087) on ice. Cells were spun down at 80 g for 15 minutes at 4° and then resuspended in cold AMES with 4% BSA (Sigma) to a concentration of 1×10⁷ cells/ml. Prior to FACS, the cells were filtered through a 40 μm cell strainer (Falcon, 352340). FACS purification with a BD Influx System cell sorter was gated as: Alexa Fluor 488 positive but Texas Red negative RGCs were collected as surRGCs; Texas Red positive RGCs were collected as regRGCs. Individual cells were directly sorted into individual wells of a 96 well-plate with 4 μl pre-filled cell lysis buffer, containing 1 U/μl of Recombinant RNase inhibitor (Clontech), 0.1% Triton X-100 (Thermo), 2.5 mM dNTP (Thermo), and 2.5 μM oligodT30VN (5′AAGCAGTGGTATCAACGCAGAGTACT30VN-3′, IDT). Cells were immediately spun down after sorting and frozen at −80° C.

Smart-seq2. Two 384-well plates containing surRGCs or regRGCs were used to generate Smart-Seq2 libraries following the published protocol at Stanford Genomics core facility and Chan Zuckerberg Biohub at Stanford. Briefly, Smartscribe (Clontech) was used for reverse transcription of the mRNAs, and then amplified by 23 PCR cycles using a KAPA Hifi HotStart Kit (Roche). Amplified cDNAs were purified by beads cleanup using a Biomek FX automated platform (Beckman), and aliquots run on a Fragment Analyzer (Agilent) for quantitation. Barcoded sequencing libraries were made using the NexteraXT DNA Library Preparation Kit (Illumina), and the PCR was performed as follows: 72° C. 3 min, 95° C. 30 s, then 10 cycles of (95° C. 10 s, 55° C. 30 s, 72° C. 1 min), 72° C. 5 min. Libraries were cleaned up by 0.8× AMPure XP beads (Beckman Coulter), then diluted to a final concentration of 2 nM for sequencing. 384 cells were sequenced in one lane of the Illumina HiSeq 4000 sequencer (Illumina) with 2× 150 bp paired-end configuration.

Data analysis: Raw data were trimmed by trim-galore (version 0.6.7) to remove adaptor sequences and aligned with Hisat2 (version 2.2.1) to the mouse reference genome (mm10). Transcripts per million reads (TPMs) per gene was calculated by StringTie (version 2.1.7), and genes with TPM>0 were defined as detected genes. All the downstream analysis was performed by R, using Seurat (version 4.1.0) with modifications. Cells with Rbpms, Slc17a6, Sncg, Thy1, Pou4f1, Pou4f2 and Pou4f3, and low Pten expression (TPM<30) were used for downstream analysis. We filtered out the low-quality cell libraries, only keeping those with (1) percentage of mitochondrial genes, <15%; (2) number of unique genes, >900; (3) total reads mapped >530,000. In total, 630 RGCs (340 regRGCs and 290 surRGCs) were selected for further analysis. We applied the Leiden algorithm (resolution=0.2) to perform unsupervised clustering of the RGCs using the TPM matrix as input, and obtained five distinct clusters. Differentially expressed genes (DEGs) in each cluster were identified using FindAllMarkers function with default parameters. The top 10 signature genes in each cluster were visualized using heatmap. Gene Ontology (GO) analysis on the DEGs was performed using R package clusterProfiler. We used Cnetplot to depict the linkage of genes and biological process as a network, showing genes in those most significant enriched terms.

For pseudo-bulk comparison of regRGCs and surRGCs, we used the FindMarkers function, which is implemented within the Seurat, to get DEGs between regRGCs and surRGCs. The DE test was performed based on the Wilcoxon rank-sum test with default parameters. The GO enrichment analysis and Cnetplot were also performed on the DEGs.

Cell cycle regression was performed using CellCycleScoring function to obtain S and G2/M phase scores for RGCs, then we predicated the classification of each RGCs to G2M, S or G1 based on the score. All raw datasets will be shared online via the Gene Expression Omnibus (GEO) database upon publication.

ON clearance and axon regeneration quantification. The CTB anterograde labeled optic nerve was trimmed and cleared by a modified iDISCO method: PBS for 30 mins; a series of 20%, 40%, 60%, 80%, and 100% methanol in 1×PBS for 30 mins at each concentration; dichloromethane (DCM)/methanol (2:1) for 30 mins; 100% DCM for 30 mins and dibenzyl ether (DBE) for 30 mins. The cleared ON was mounted on slides between two 22×22 mm cover slips supported with DBE, covered with a 22×22 mm cover slip, and sealed with clear nail polish. The mounted whole nerve was imaged with a 25× oil immersion objective lens, using the airy scan mode (6 μm per stack), Z stack and tile scan. The number of CTB labeled axons was quantified as described previously. Briefly, we counted the fibers that crossed perpendicular lines drawn on the ON optical sections distal to the crush site at 250, 500, 1000, 1500, and then every 250 μm till no fibers were visible. 3 Z-stacks at depths of 60, 120 and 180 μm were sampled to acquire the mean axon density of the ON, (axon number)/(R*t). The width of the stack (R) was measured at the point (d) at which the counts were taken and used together with the thickness of the optical section (t=6 μm) to calculate the number of axons/μm² area of the stack. The mean axon density of the 3 stacks was used to calculate the total axon number, Σad=πr²* mean axon density, r is the radius of the ON. All CTB signals that were in the range of intensity that was set from lowest intensity to the maximum intensity after background subtraction were counted as individual fibers. The investigators who counted the cells or axons were blinded to the treatment of the samples.

Brain-ON clearance and light sheet microscopy imaging. The attached ON and whole brain were carefully dissected with fine forceps and scissors, and embedded in 1.5% agarose gel block. The tissue embedded gel block was cleared with modified iDISCO method: PBS for 4 hours; a series of 20%, 40%, 60%, 80%, and 100% methanol in 1×PBS for 1 day at each concentration; dichloromethane (DCM)/methanol (2:1) for 1 day; 100% DCM for 1 day and dibenzyl ether (DBE) for 1 day. The ventral side of the tissue gel block was faced up and fixed on a spike holder, then placed into the imaging chamber immersed in the DBE buffer. The ultramicroscope II generated 6 bi-directional 3.89 μm thin light sheets to illuminate the tissue gel block from both sides while imaging the excited plane with a 2× objective microscope perpendicular to the sample using a 0.63× zoom for whole tissue and a 6.3× zoom for regenerating axons. Tissue was imaged with the diode 561 nm laser, emission filter 620/60 nm and sheet numerical aperture (NA) 0.149 through a 2 μm step-size of the Z-stack. The multiple optical sliced images of the whole tissue were collected and further maximum projections were processed by Fiji/Image J.

In vivo EdU labeling of cell proliferation in retina. We followed the procedure described in a previous publication. The Pten KO and WT mice were anesthetized with xylazine and ketamine based on their body weight (0.01 mg xylazine/g+0.08 mg ketamine/g). Intravitreal injection of 2 μl 1 mg/ml EdU-Alexa 488 (ThermoFisher, C10337) was performed at 3dpc, 7dpc, 10dpc, and 14dpc (4 hours before sacrificing). Mice were sacrificed at 14dpc for immunostaining.

SOHU glaucoma model and IOP measurement. The detailed procedure has been published before. In brief, 9 week old mice were anesthetized by an intraperitoneal injection of Avertin (0.3 mg/g) before a tunnel was made by a 32G needle through the layers of the cornea on the superotemporal side close to the limbus to reach the anterior chamber without injuring lens or iris. Then, ˜2 μl silicone oil (1,000 mPa·s, Silikon, Alcon Laboratories, Fort Worth, Texas) was injected slowly into the anterior chamber using a homemade sterile glass micropipette, until the oil droplet expanded to cover most areas of the iris (diameter ˜1.8-2.2 mm). After the injection, veterinary antibiotic ointment (BNP Ophthalmic Ointment, Vetropolycin, Dechra, Overland Park, Kansas) was applied to the surface of the injected eye. The contralateral control eyes received 2 μl normal saline to the anterior chamber. Throughout the procedure, artificial tears (Systane Ultra Lubricant Eye Drops, Alcon Laboratories, Fort Worth, Texas) were applied to keep the cornea moist. The IOP of both eyes was measured before SO injection and at 3wpi by the TonoLab tonometer (Colonial Medical Supply, Espoo, Finland) according to product instructions. Briefly, in the morning, mice were anesthetized with a sustained flow of isoflurane (3% isoflurane at 2 L/minute mixed with oxygen) delivered to the nose by a special rodent nose cone (Xenotec, Inc., Rolla, Missouri), which left the eyes exposed for IOP measurement. 1% Tropicamide sterile ophthalmic solution (Akorn, Somerset, New Jersey) was applied three times at 3-minute intervals to fully dilate the pupils (about 10 minutes) before taking measurements. The average of six measurements by the TonoLab was considered as one machine-generated reading and three machine-generated readings were obtained from each eye; the mean was calculated to determine the IOP. During this procedure, artificial tears were applied to keep the cornea moist.

Immunohistochemistry of retinal wholemounts and RGC counts. After transcardiac perfusion with 4% PFA in PBS, the eyes were dissected out, post-fixed with 4% PFA for 2 hours, at room temperature, and cryoprotected in 30% sucrose overnight. Retinas were then dissected out and washed extensively in PBS before blocking in staining buffer (10% normal donkey serum and 2% Triton X-100 in PBS) for 30 minutes. RBPMS guinea pig antibody (ProSci, California), HA antibody and AP2a mouse antibody were used at 1:4000, 1:500, and 1:100 to label RGCs and amacrine cells, respectively. Floating retinas were incubated with primary antibodies overnight at 4° C. and washed 3 times for 30 minutes each with PBS. Secondary antibodies (Alexa Fluor 647-goat anti-guinea pig, Cy3-goat anti-rat and Cy2-goat anti-mouse) were then applied (1:200; Jackson ImmunoResearch, West Grove, Pennsylvania) and incubated for 1 hour at room temperature. Retinas were again washed 3 times for 30 minutes each with PBS before a cover slip was attached with Fluoromount-G (Southernbiotech, Birmingham, Alabama). Images of immunostained wholemounts were acquired with a Zeiss M2 epifluorescence microscope and Zeiss confocal microscope (LSM 880) with 20× and 40× oil lens and serial filters (BP410-510 for DAPI or Fast Blue, BP520-550 for Alexa Fluor 488, Dextran-FITC or Cy2, BP565-650 for Cy3 or Dextran Texas red and BP650-750 for Alexa Fluor 647). For RGC counting, 6-9 fields of 332 μm×332 μm area were sampled on average from peripheral or middle and central regions of each whole retina for imaging and stitching by a 20× lens and a Keyence fluorescence microscope (Itasca, BZ-X800), and RBPMS⁺ RGCs counted by Fiji/Image J (NIH). The percentage of RGC survival was calculated as the ratio of surviving RGC numbers in injured eyes compared to contralateral uninjured eyes. The investigators who counted the cells were blinded to the treatment of the samples.

ON semi-thin sections and quantification of surviving axons. The detailed procedure has been described previously. Briefly, transverse semi-thin (1 μm) sections of ON were cut on an ultramicrotome (EM UC7, Leica, Wetzlar, Germany) from tissue collected 2 mm distal to the eye (about 1.5 mm distal to the crush site) and stained with 1% para-phenylenediamine (PPD) in methanol:isopropanol (1:1). Whole ONs were imaged and stitched through a 100× lens of a Keyence fluorescence microscopy. Eight areas of 21.4 μm×29.1 μm from the entire ON were cropped on average and counted manually with Fiji/ImageJ. After counting all the images taken from a single nerve, the mean of the surviving axon number was calculated for each ON, and compared to that in the contralateral control ON to yield a percentage of axon survival value. The investigators who counted the axons were masked to the treatment of the samples.

Spectral-domain optical coherence tomography (SD-OCT) imaging and scanning laser ophthalmoscopy (SLO) fundus imaging. Fundus OCT imaging was performed under OCT mode by switching to a 30° licensed lens (Heidelberg Engineering) as in the previously described procedure. Briefly, the mouse retina was scanned with the ring scan mode centered by the ON head at 100 frames average under high-resolution mode (each B-scan consisted of 1536 A scans). The ganglion cell complex (GCC) includes retinal nerve fiber layer (RNFL), ganglion cell layer (GCL), and inner plexiform layer (IPL). The average thickness of GCC around the ON head was measured manually with the Heidelberg software. The mean of the GCC thickness in the injured retina was compared to that in the contralateral control retina to yield a percentage of GCC thickness value. The investigators who measured the thickness of GCC were blinded to the treatment of the samples. For SLO retinal fundus imaging, the fundus labeled with green fluorescent dye was imaged under FA mode by switching to a 55° non-contact lens and a customized +10D contact lens (3.0 mm diameter, 1.6 mm BC, PMMA clear, Advanced Vision Technologies) (Heidelberg Engineering) as previously described. The mouse retina was imaged under the high-resolution mode (1536×1536 pixels) and 100 frames average with 488 nm excitation laser.

Pattern electroretinogram (PERG) recording. PERG recording of both eyes was performed at the same time with the Miami PERG system (Intelligent Hearing Systems, Miami, FL), as described in our previous publication. In brief, a feedback-controlled heating pad (TCAT-2LV, Physitemp Instruments Inc., Clifton, New Jersey) maintained animal core temperature at 37° C. A small lubricant eye drop (Systane) was applied before recording to prevent corneal opacities. The reference electrode was placed subcutaneously on the back of the head between the two ears, the ground electrode was placed at the root of the tail and the active steel needle electrode was placed subcutaneously on the snout for the simultaneous acquisition of left and right eye responses. Two 14 cm×14 cm LED-based stimulators were placed in front so that the center of each screen was 10 cm from each eye. The pattern remained at a contrast of 85% and a luminance of 800 cd/m², and consisted of four cycles of black-gray elements, with a spatial frequency of 0.052 c/d. Upon stimulation, the independent PERG signals were recorded from the snout and simultaneously by asynchronous binocular acquisition. With each trace recording up to 1020 ms, two consecutive recordings of 200 traces were averaged to achieve one readout. The first positive peak in the waveform was designated as P1 and the second negative peak as N2. P1 was typically around 100 ms. The mean amplitude of the P1-N2 amplitude in the injured eye was compared to that in the contralateral control eye to yield a percentage of amplitude change. The investigators who measured the amplitudes were blinded to the treatment of the samples.

OKR measurement. The spatial vision of both eyes was measured using the opto-kinetic response (OKR) as described in our previous publication^{57,59}. In brief, living mice were placed unrestrained on a platform in the center of four 17-inch LCD computer monitors (Dell, Phoenix, AZ); their movement was captured by a video camera above the platform. A rotating cylinder with vertical sine wave grating was computed and projected to the four monitors by OptoMotry software (CerebralMechanics Inc., Lethbridge, Alberta, Canada). The sine wave grating provides a virtual-reality environment to measure the spatial acuity of left eye when rotated clockwise and right eye when rotated counterclockwise. When the mouse calmed down and stopped moving, the gray of the monitor immediately switched to a low spatial frequency (0.1 cycle/degree) for five seconds, in which the mouse was assessed by judging whether the head turned to track the grating. The short time frame of assessment ensured that the mice did not adapt to the stimulus, which would lead to false readouts. The mice were judged to be capable of tracking the grating. The spatial frequency was increased repeatedly until a maximum frequency was identified and recorded. The % of vision acuity was yielded by comparing the maximum frequency of the experimental eye to that of the contralateral eye. The mice were tested in the morning and the investigator who judged the OKR was blinded to the treatment of the mice.

Statistical analyses. GraphPad Prism 6 was used to generate graphs and for statistical analyses. Data are presented as means±s.e.m. Student's t-test was used for two groups comparison, One-way ANOVA with post hoc test and Two-way ANOVA were used for multiple comparisons.

### Sequences

(SEQ ID NO: 1) mSncg-1.45 kb promoter
ccattgacgtcaataatgacgtatgttcccatagtaacgccaatagggactttccattgacgtcaatgggtggagtatttacggtaaactg
cccacttggcagtacatcaagtgtatcatatgggtcccatgccactagtgggagctgtgttacctgttgcagccccacccaaagcccct
gctataggtcaagcaggaatcaccctgccatccccagcctggggcctggagtaccagatccaggaaactagcatcccttagctatag
agatagccacacatcagcccattcctcagatgtgtatctggggctcagacatcatctcccgatctccgacaagggcaggatttccttac
cgtctgatggggtctctgctggtatcctcagcccctagtctccagccttcaggcatgccgggcctattgagatgggagaacttggtaccg
gggtcctgtgcccaggaccctagcagtccccagctcaggtacaccccaaagcccagcagcagtgtogggatcatggtgaggggct
cctgtgctgatgctcagccttacaagtgactctcaaatttgctggtgatgtggtcttcaagcgaaatgtcagaaagaaaagaaaacacg
aggacaacaaagggaggaagtggcctggtccggcccacccggcaagtctcatccgcccccgcccccgccccttccagcetggcc
cccttggaggcctccaaccactcaggtcaattcctgtgtcctgagggcacttgaatcagggacacgggatttggtagacacataaagg
tggccccattaaacttatttctccaggactctgtcgtgggcctggaggagatctggtgccacccatactgttggccaggaagtggggaa
cgggcacatcacacctgctcggcaccttgggctatgggaactagcaggtgggtgggaactcagagaaggaaagggactatgctag
aatcacacagcgggcagcccagtctagggcatggggagcagctttgggtgtttctggcctcagccttccaacaggtttggctagagct
ccaggctcaagagcatccaggatacagtggggaactggataacagggcagcctgcaggttggccattcattggattggccctgacc
ccggcccagcctgggacactgaggcatcatcagtcaaggcacttttcttctgcatataagagccagggcacgagaccaccagggctt
tccaaggatgaatgaggtgtaatgatagattaggatatgtccagcctccaacacgctctccctcccccagggccaacaagagtcagc
agggcagaatagagccagtaggggcccgggccctgctcgctggtatccccgtgaggcatgccttctctctggcccgccctccctgcc
cccaccctggcccgggctggctgggctccagccagcagccacagcatcaatatttcatctgcgtcaataagaggcagtagcagcag
agacagcggctgcggcagcactccagtccatagcttgcagcagccaggttccatccttgcaaacaccatggacgtcttcaag
(SEQ ID NO: 2) mSncg-1.03 b promoter
ccattgacgtcaataatgacgtatgttcccatagtaacgccaatagggactttccattgacgtcaatgggtggagtatttacggtaaactg
cccacttggcagtacatcaagtgtatcatatgtcagccttacaagtgactctcaaatttgctggtgatgtggtcttcaagcgaaatgtcaga
aagaaaagaaaacacgaggacaacaaagggaggaagtggcctggtccggcccacccggcaagtctcatccgcccccgccccc
gccccttccagcctggcccccttggaggcctccaaccactcaggtcaattcctgtgtcctgagggcacttgaatcagggacacgggatt
tggtagacacataaaggtggccccattaaacttatttctccaggactctgtcgtgggcctggaggagatctggtgccacccatactgttg
gccaggaagtggggaacgggcacatcacacctgctcggcaccttgggctatgggaactagcaggtgggtgggaactcagagaag
gaaagggactatgctagaatcacacagcgggcagcccagtctagggcatggggagcagctttgggtgtttctggcctcagccttcca
acaggtttggctagagctccaggctcaagagcatccaggatacagtggggaactggataacagggcagcctgcaggttggccattc
attggattggccctgaccccggcccagcctgggacactgaggcatcatcagtcaaggcacttttcttctgcatataagagccagggca
cgagaccaccagggctttccaaggatgaatgaggtgtaatgatagattaggatatgtccagcctccaacacgctctccctcccccagg
gccaacaagagtcagcagggcagaatagagccagtaggggcccgggccctgctcgctggtatccccgtgaggcatgccttctctct
ggcccgccctccctgcccccaccctggcccgggctggctgggctccagccagcagccacagcatcaatatttcatctgcgtcaataa
gaggcagtagcagcagagacagcggctgcggcagcactccagtccatagcttgcagcagccaggttccatccttgcaaacaccat
ggacgtcttcaag
(SEQ ID NO: 3) mSncg-0.66 kb promoter
ccattgacgtcaataatgacgtatgttcccatagtaacgccaatagggactttccattgacgtcaatgggtggagtatttacggtaaactg
cccacttggcagtacatcaagtgtatcatatgctatgggaactagcaggtgggtgggaactcagagaaggaaagggactatgctaga
atcacacagcgggcagcccagtctagggcatggggagcagctttgggtgtttctggcctcagccttccaacaggtttggctagagctcc
aggctcaagagcatccaggatacagtggggaactggataacagggcagcctgcaggttggccattcattggattggccctgacccc
ggcccagcctgggacactgaggcatcatcagtcaaggcacttttcttctgcatataagagccagggcacgagaccaccagggctttc
caaggatgaatgaggtgtaatgatagattaggatatgtccagcctccaacacgctctccctcccccagggccaacaagagtcagca
gggcagaatagagccagtaggggcccgggccctgctcgctggtatccccgtgaggcatgccttctctctggcccgccctccctgcccc
caccctggcccgggctggctgggctccagccagcagccacagcatcaatatttcatctgcgtcaataagaggcagtagcagcagag
acagcggctgcggcagcactccagtccatagottgcagcagccaggttccatccttgcaaacaccatggacgtcttcaag
(SEQ ID NO: 4) mSncg-0.27 kb promoter
ccattgacgtcaataatgacgtatgttcccatagtaacgccaatagggactttccattgacgtcaatgggtggagtatttacggtaaactg
cccacttggcagtacatcaagtgtatcatatgaagagtcagcagggcagaatagagccagtaggggcccgggccctgctcgctggt
atccccgtgaggcatgccttctctctggcccgccctccctgcccccaccctggcccgggctggctgggctccagccagcagccacag
catcaatatttcatctgcgtcaataagaggcagtagcagcagagacagcggctgcggcagcactccagtccatagcttgcagcagcc
aggttccatccttgcaaacaccatggacgtcttcaag
(SEQ ID NO: 5) human ANXA2 protein
VKAYTNFDAERDALNIETAIKTKGVDEVTIVNILTNRSNAQRQDIAFAYQRRTKKELASALKSALS
GHLETVILGLLKTPAQYDASELKASMKGLGTDEDSLIEIICSRTNQELQEINRVYKEMYKTDLEKD
IISDTSGDFRKLMVALAKGRRAEDGSVIDYELIDQDARDLYDAGVKRKGTDVPKWISIMTERSVP
HLQKVFDRYKSYSPYDMLESIRKEVKGDLENAFLNLVQCIQNKPLYFADRLYDSMKGKGTRDK
VLIRIMVSRSEVDMLKIRSEFKRKYGKSLYYYIQQDTKGDYQKALLYLCGGDD″
(SEQ ID NO: 6) human ANXA2 coding sequence
gctcagcatt tggggacgct ctcagctctc ggcgcacggc ccaggtaagc ggggcgcgcc ctgcccgccc gcgatgggcc
gccagctagc ggggtgtgga gacgctggga agaaggcttc cttcaaaatg tctactgttc acgaaatcct gtgcaagctc
agcttggagg gtgatcactc tacaccccca agtgcatatg ggtctgtcaa agcctatact aactttgatg ctgagcggga
tgctttgaac attgaaacag ccatcaagac caaaggtgtg gatgaggtca ccattgtcaa cattttgacc aaccgcagca
atgcacagag acaggatatt gccttcgcct accagagaag gaccaaaaag gaacttgcat cagcactgaa gtcagcctta
tctggccacc tggagacggt gattttgggc ctattgaaga cacctgctca gtatgacgct tctgagctaa aagcttccat
gaaggggctg ggaaccgacg aggactctct cattgagatc atctgctcca gaaccaacca ggagctgcag gaaattaaca
gagtctacaa ggaaatgtac aagactgatc tggagaagga cattatttcg gacacatctg gtgacttccg caagctgatg
gttgccctgg caaagggtag aagagcagag gatggctctg tcattgatta tgaactgatt gaccaagatg ctcgggatct
ctatgacgct ggagtgaaga ggaaaggaac tgatgttccc aagtggatca gcatcatgac cgagcggagc gtgccccacc
tccagaaagt atttgatagg tacaagagtt acagccctta tgacatgttg gaaagcatca ggaaagaggt taaaggagac
ctggaaaatg ctttcctgaa cctggttcag tgcattcaga acaagcccct gtattttgct gatcggctgt atgactccat
gaagggcaag gggacgcgag ataaggtcct gatcagaatc atggtctccc gcagtgaagt ggacatgttg aaaattaggt
ctgaattcaa gagaaagtac ggcaagtccc tgtactatta tatccagcaa gacactaagg gcgactacca gaaagcgctg
ctgtacctgt gtggtggaga tgactgaagc ccgacacggc ctgagcgtcc agaaatggtg ctcaccatgc ttccagctaa
caggtctaga aaaccagctt gcgaataaca gtccccgtgg ccatccctgt gagggtgacg ttagcattac ccccaacctc
attttagttg cctaagcatt gcctggcctt cctgtctagt ctctcctgta agccaaagaa atgaacattc caaggagttg
gaagtgaagt ctatgatgtg aaacactttg cctcctgtgt actgtgtcat aaacagatga ataaactgaa tttgtacttt
agaaacacgt actttgtggc cctgctttca actgaattgt ttgaaaatta aacgtgcttg gggttcagct ggtgaggctg
tccctgtagg aagaaagctc tgggactgag ctgtacagta tggttgcccc tatccaagtg tcgctattta agttaaattt
aaatgaaata aaataaaata aaatca
(SEQ ID NO: 7) human tissue plasminogen activator protein
MDAMKRGLCCVLLLCGAVFVSPSQEIHARFRRGARSYQVICRDEKTQMIYQQHQSWLRPVLR
SNRVEYCWCNSGRAQCHSVPVKSCSEPRCFNGGTCQQALYFSDFVCQCPEGFAGKCCEIDT
RATCYEDQGISYRGTWSTAESGAECTNWNSSALAQKPYSGRRPDAIRLGLGNHNYCRNPDR
DSKPWCYVFKAGKYSSEFCSTPACSEGNSDCYFGNGSAYRGTHSLTESGASCLPWNSMILIG
KVYTAQNPSAQALGLGKHNYCRNPDGDAKPWCHVLKNRRLTWEYCDVPSCSTCGLRQYSQP
QFRIKGGLFADIASHPWQAAIFAKHRRSPGERFLCGGILISSCWILSAAHCFQERFPPHHLTVIL
GRTYRVVPGEEEQKFEVEKYIVHKEFDDDTYDNDIALLQLKSDSSRCAQESSVVRTVCLPPADL
QLPDWTECELSGYGKHEALSPFYSERLKEAHVRLYPSSRCTSQHLLNRTVTDNMLCAGDTRS
GGPQANLHDACQGDSGGPLVCLNDGRMTLVGIISWGLGCGQKDVPGVYTKVTNYLDWIRDN
MRP
(SEQ ID NO: 8) human tissue plasminogen activator coding sequence
agagctgaga tcctacagga gtccagggct ggagagaaaa cctctgcgag gaaagggaag gagcaagccg tgaatttaag
ggacgctgtg aagcaatcat ggatgcaatg aagagagggc tctgctgtgt gctgctgctg tgtggagcag tcttcgtttc
gcccagccag gaaatccatg cccgattcag aagaggagcc agatcttacc aagtgatctg cagagatgaa aaaacgcaga
tgatatacca gcaacatcag tcatggctgc gccctgtgct cagaagcaac cgggtggaat attgctggtg caacagtggc
agggcacagt gccactcagt gcctgtcaaa agttgcagcg agccaaggtg tttcaacggg ggcacctgcc agcaggccct
gtacttctca gatttcgtgt gccagtgccc cgaaggattt gctgggaagt gctgtgaaat agataccagg gccacgtgct
acgaggacca gggcatcagc tacaggggca cgtggagcac agcggagagt ggcgccgagt gcaccaactg
gaacagcagc gcgttggccc agaagcccta cagcgggcgg aggccagacg ccatcaggct gggcctgggg aaccacaact
actgcagaaa cccagatcga gactcaaagc cctggtgcta cgtctttaag gcggggaagt acagctcaga gttctgcagc
acccctgcct gctctgaggg aaacagtgac tgctactttg ggaatgggtc agcctaccgt ggcacgcaca gcctcaccga
gtcgggtgcc tcctgcctcc cgtggaattc catgatcctg ataggcaagg tttacacagc acagaacccc agtgcccagg
cactgggcct gggcaaacat aattactgcc ggaatcctga tggggatgcc aagccctggt gccacgtgct gaagaaccgc
aggctgacgt gggagtactg tgatgtgccc tcctgctcca cctgcggcct gagacagtac agccagcctc agtttcgcat
caaaggaggg ctcttcgccg acatcgcctc ccacccctgg caggctgcca tctttgccaa gcacaggagg tcgcccggag
agcggttcct gtgcgggggc atactcatca gctcctgctg gattctctct gccgcccact gcttccagga gaggtttccg
ccccaccacc tgacggtgat cttgggcaga acataccggg tggtccctgg cgaggaggag cagaaatttg aagtcgaaaa
atacattgtc cataaggaat tcgatgatga cacttacgac aatgacattg cgctgctgca gctgaaatcg gattcgtccc
gctgtgccca ggagagcagc gtggtccgca ctgtgtgcct tcccccggcg gacctgcagc tgccggactg gacggagtgt
gagctctccg gctacggcaa gcatgaggcc ttgtctcctt tctattcgga gcggctgaag gaggctcatg tcagactgta
cccatccagc cgctgcacat cacaacattt acttaacaga acagtcaccg acaacatgct gtgtgctgga gacactcgga
gcggcgggcc ccaggcaaac ttgcacgacg cctgccaggg cgattcggga ggccccctgg tgtgtctgaa cgatggccgc
atgactttgg tgggcatcat cagctggggc ctgggctgtg gacagaagga tgtcccgggt gtgtacacca aggttaccaa
ctacctagac tggattcgtg acaacatgcg accgtgacca ggaacacccg actcctcaaa agcaaatgag atcccgcctc
ttcttcttca gaagacactg caaaggcgca gtgcttctct acagacttct ccagacccac cacaccgcag aagcgggacg
agaccctaca ggagagggaa gagtgcattt tcccagatac ttcccatttt ggaagttttc aggacttggt ctgatttcag
gatactctgt cagatgggaa gacatgaatg cacactagcc tctccaggaa tgcctcctcc ctgggcagaa agtggccatg
ccaccctgtt ttcagctaaa gcccaacctc ctgacctgtc accgtgagca gctttggaaa caggaccaca aaaatgaaag
catgtctcaa tagtaaaaga taacaagatc tttcaggaaa gacggattgc attagaaata gacagtatat ttatagtcac
aagagcccag cagggcctca aagttggggc aggctggctg gcccgtcatg ttcctcaaaa gcacccttga cgtcaagtct
ccttcccctt tccccactcc ctggctctca gaaggtattc cttttgtgta cagtgtgtaa agtgtaaatc ctttttcttt
ataaacttta
gagtagcatg agagaattgt atcatttgaa caactaggct tcagcatatt tatagcaatc catgttagtt tttactttct
gttgccacaa
ccctgtttta tactgtactt aataaattca gatatatttt tcacagtttt tccaaaatca gagtggaatg gttttgttat
agatgctgta
tcccactctt tattcatgtt cacattttaa aatcatttgg aattctgctt cactcgctta acatatacac aacacctgta
acatacaagg
caatgggcta ggtgctccag accgggaaaa ggagggacag gaatgcttgg tctgatgggc taatatggca tttagagaag
taccaaggta cagtggagcc ggtcacaaaa gggcagactt gtagtagaat tcagttgcaa gagggattgg ggaatcttaa
ggaaaaaata gaatcttaag gaaaaaataa ctgggtgaga cgtggactgt ggacaggtgt ggaaaaggca ctctccatgg
aggtatgaat atgtagaggg ccaagagagg ggagtacagg gagaaatgag ttgagcttgt ctgaagtgaa cttcaggaag
aggaacatag gctggaattt agattatggg ggctctgaac accaaactga gtttggactt aattgacttc tg

Quigley, H. A. et al. Retinal ganglion cell death in experimental glaucoma and after axotomy occurs by apoptosis. Invest Ophthalmol Vis Sci 36, 774-786 (1995).
Libby, R. T. et al. Susceptibility to neurodegeneration in a glaucoma is modified by Bax gene dosage. PLoS Genet 1, 17-26, doi: 10.1371/journal.pgen.0010004 (2005).
Howell, G. R. et al. Axons of retinal ganglion cells are insulted in the optic nerve early in DBA/2J glaucoma. J Cell Biol 179, 1523-1537, doi: 10.1083/jcb.200706181 (2007).
Nickells, R. W., Howell, G. R., Soto, I. & John, S. W. Under pressure: cellular and molecular responses during glaucoma, a common neurodegeneration with axonopathy. Annu Rev Neurosci 35, 153-179, doi: 10.1146/annurev.neuro.051508.135728 (2012).
Jonas, J. B. et al. Glaucoma. Lancet 390, 2183-2193, doi: 10.1016/S0140-6736 (17) 31469-1 (2017).
Weinreb, R. N. et al. Primary open-angle glaucoma. Nat Rev Dis Primers 2, 16067, doi: 10.1038/nrdp.2016.67 (2016).
Calkins, D. J. Adaptive responses to neurodegenerative stress in glaucoma. Prog Retin Eye Res, 100953, doi: 10.1016/j.preteyeres.2021.100953 (2021).
Tham, Y. C. et al. Global prevalence of glaucoma and projections of glaucoma burden through 2040: a systematic review and meta-analysis. Ophthalmology 121, 2081-2090, doi: 10.1016/j.ophtha.2014.05.013 (2014).
Steinmetz, J. D. et al. Causes of blindness and vision impairment in 2020 and trends over 30 years, and prevalence of avoidable blindness in relation to VISION 2020: the Right to Sight: an analysis for the Global Burden of Disease Study. The Lancet Global Health 9, e144-e160, doi: 10.1016/s2214-109x(20) 30489-7 (2021).
Burgoyne, C. F. A biomechanical paradigm for axonal insult within the optic nerve head in aging and glaucoma. Experimental eye research 93, 120-132, doi: 10.1016/j.exer.2010.09.005 (2011).
Stowell, C. et al. Biomechanical aspects of axonal damage in glaucoma: A brief review. Experimental eye research 157, 13-19, doi: 10.1016/j.exer.2017.02.005 (2017).
Heijl, A. et al. Reduction of intraocular pressure and glaucoma progression: results from the Early Manifest Glaucoma Trial. Arch Ophthalmol 120, 1268-1279, doi: 10.1001/archopht.120.10.1268 (2002).
Garway-Heath, D. F. et al. Latanoprost for open-angle glaucoma (UKGTS): a randomised, multicentre, placebo-controlled trial. Lancet 385, 1295-1304, doi: 10.1016/S0140-6736 (14) 62111-5 (2015).
Anderson, D. R. & Normal Tension Glaucoma, S. Collaborative normal tension glaucoma study. Curr Opin Ophthalmol 14, 86-90, doi: 10.1097/00055735-200304000-00006 (2003).
Wormald, R., Virgili, G. & Azuara-Blanco, A. Systematic reviews and randomised controlled trials on open angle glaucoma. Eye (Lond) 34, 161-167, doi: 10.1038/s41433-019-0687-5 (2020).
Park, K. K. et al. Promoting axon regeneration in the adult CNS by modulation of the PTEN/mTOR pathway. Science 322, 963-966, doi: 10.1126/science. 1161566 (2008).
Williams, P. R., Benowitz, L. I., Goldberg, J. L. & He, Z. Axon Regeneration in the Mammalian Optic Nerve. Annual review of vision science 6, 195-213, doi: 10.1146/annurev-vision-022720-094953 (2020).
Zhang, J., Yang, D., Huang, H., Sun, Y. & Hu, Y. Coordination of Necessary and Permissive Signals by PTEN Inhibition for CNS Axon Regeneration. Front Neurosci 12, 558, doi: 10.3389/fnins.2018.00558 (2018).
Liu, K. et al. PTEN deletion enhances the regenerative ability of adult corticospinal neurons. Nat Neurosci 13, 1075-1081, doi: 10.1038/nn.2603 (2010).
Jin, D. et al. Restoration of skilled locomotion by sprouting corticospinal axons induced by co-deletion of PTEN and SOCS3. Nature communications 6, 8074, doi: 10.1038/ncomms9074 (2015).
Song, Y. et al. Regeneration of Drosophila sensory neuron axons and dendrites is regulated by the Akt pathway involving Pten and microRNA bantam. Genes Dev 26, 1612-1625, doi: 10.1101/gad.193243.112 (2012).
Byrne, A. B. et al. Insulin/IGF1 Signaling Inhibits Age-Dependent Axon Regeneration. Neuron 81, 561-573, doi: 10.1016/j.neuron.2013.11.019 (2014).
Zoncu, R., Efeyan, A. & Sabatini, D. M. mTOR: from growth signal integration to cancer, diabetes and ageing. Nat Rev Mol Cell Biol 12, 21-35, doi: 10.1038/nrm3025 (2011).
Yang, L. et al. The mTORC1 effectors S6K1 and 4E-BP play different roles in CNS axon regeneration. Nature communications 5, 5416, doi: 10.1038/ncomms6416 (2014).
Miao, L. et al. mTORC1 is necessary but mTORC2 and GSK3beta are inhibitory for AKT3-induced axon regeneration in the central nervous system. eLife 5, e14908, doi: 10.7554/eLife.14908 (2016).
Huang, H. et al. AKT-dependent and -independent pathways mediate PTEN deletion-induced CNS axon regeneration. Cell death & disease 10, 203, doi: 10.1038/s41419-018-1289-z (2019).
Hu, Y. The necessary role of mTORC1 in central nervous system axon regeneration. Neural regeneration research 10, 186-188, doi: 10.4103/1673-5374.152363 (2015).
Tanay, A. & Regev, A. Scaling single-cell genomics from phenomenology to mechanism. Nature 541, 331-338, doi: 10.1038/nature21350 (2017).
Regev, A. et al. The Human Cell Atlas. eLife 6, doi: 10.7554/eLife.27041 (2017).
Tran, N. M. et al. Single-Cell Profiles of Retinal Ganglion Cells Differing in Resilience to Injury Reveal Neuroprotective Genes. Neuron, doi: 10.1016/j.neuron.2019.11.006 (2019).
Yan, W. et al. Cell Atlas of The Human Fovea and Peripheral Retina. Scientific reports 10, 9802, doi: 10.1038/s41598-020-66092-9 (2020).
Clark, B. S. et al. Single-Cell RNA-Seq Analysis of Retinal Development Identifies NFI Factors as Regulating Mitotic Exit and Late-Born Cell Specification. Neuron 102, 1111-1126 e1115, doi: 10.1016/j.neuron.2019.04.010 (2019). Hoang, T. et al. Gene regulatory networks controlling vertebrate retinal regeneration. Science 370, doi: 10.1126/science.abb8598 (2020).
Picelli, S. et al. Smart-seq2 for sensitive full-length transcriptome profiling in single cells. Nature methods 10, 1096-1098, doi: 10.1038/nmeth.2639 (2013).
Picelli, S. et al. Full-length RNA-seq from single cells using Smart-seq2. Nature protocols 9, 171-181, doi: 10.1038/nprot.2014.006 (2014).
Macosko, E. Z. et al. Highly Parallel Genome-wide Expression Profiling of Individual Cells Using Nanoliter Droplets.
Cell 161, 1202-1214, doi: 10.1016/j.cell.2015.05.002 (2015).
Shekhar, K. et al. Comprehensive Classification of Retinal Bipolar Neurons by Single-Cell Transcriptomics. Cell
166, 1308-1323 e1330, doi: 10.1016/j.cell.2016.07.054 (2016).
Peng, Y. R. et al. Molecular Classification and Comparative Taxonomics of Foveal and Peripheral Cells in Primate Retina. Cell 176, 1222-1237 e1222, doi: 10.1016/j.cell.2019.01.004 (2019).
Daniszewski, M. et al. Single cell RNA sequencing of stem cell-derived retinal ganglion cells. Sci Data 5, 180013, doi: 10.1038/sdata.2018.13 (2018).
Hu, Y. et al. Differential effects of unfolded protein response pathways on axon injury-induced death of retinal ganglion cells. Neuron 73, 445-452, doi: 10.1016/j.neuron.2011.11.026 (2012).
Yang, L. et al. Rescue of Glaucomatous Neurodegeneration by Differentially Modulating Neuronal Endoplasmic Reticulum Stress Molecules. J Neurosci 36, 5891-5903, doi: 10.1523/JNEUROSCI.3709-15.2016 (2016).
Huang, H. et al. Neuroprotection by eIF2alpha-CHOP inhibition and XBP-1 activation in EAE/optic neuritiss. Cell death & disease 8, e2936, doi: 10.1038/cddis.2017.329 (2017).
Liu, P. et al. Neuronal RAG Overexpression Does Not Achieve Significant Neuroprotection in Experimental Autoimmune Encephalomyelitis/Optic Neuritis. Frontiers in cellular neuroscience 15, doi: 10.3389/fncel.2021.754651 (2021).
Lu, Y. et al. Reprogramming to recover youthful epigenetic information and restore vision. Nature 588, 124-129, doi: 10.1038/s41586-020-2975-4 (2020).
Wang, Q. et al. Mouse gamma-Synuclein Promoter-Mediated Gene Expression and Editing in Mammalian Retinal Ganglion Cells. J Neurosci 40, 3896-3914, doi: 10.1523/JNEUROSCI.0102-20.2020 (2020).
Mora-Castilla, S. et al. Miniaturization Technologies for Efficient Single-Cell Library Preparation for Next-Generation Sequencing. J Lab Autom 21, 557-567, doi: 10.1177/2211068216630741 (2016).
Picelli, S. Full-Length Single-Cell RNA Sequencing with Smart-seq2. Methods Mol Biol 1979, 25-44, doi: 10.1007/978-1-4939-9240-9_3 (2019).
Duan, X. et al. Subtype-specific regeneration of retinal ganglion cells following axotomy: effects of osteopontin and mTOR signaling. Neuron 85, 1244-1256, doi: 10.1016/j.neuron.2015.02.017 (2015).
Abreu, C. A., De Lima, S. V., Mendonca, H. R., Goulart, C. O. & Martinez, A. M. Absence of galectin-3 promotes neuroprotection in retinal ganglion cells after optic nerve injury. Histol Histopathol 32, 253-262, doi: 10.14670/HH-11-788 (2017).
Ohkawa, N., Fujitani, K., Tokunaga, E., Furuya, S. & Inokuchi, K. The microtubule destabilizer stathmin mediates the development of dendritic arbors in neuronal cells. Journal of cell science 120, 1447-1456, doi: 10.1242/jcs.001461 (2007).
Grenningloh, G., Soehrman, S., Bondallaz, P., Ruchti, E. & Cadas, H. Role of the microtubule destabilizing proteins SCG10 and stathmin in neuronal growth. J Neurobiol 58, 60-69, doi: 10.1002/neu.10279 (2004).
Bharadwaj, A. G., Kempster, E. & Waisman, D. M. The ANXA2/S100A10 Complex-Regulation of the Oncogenic Plasminogen Receptor. Biomolecules 11, doi: 10.3390/biom11121772 (2021).
Bharadwaj, A., Bydoun, M., Holloway, R. & Waisman, D. Annexin A2 heterotetramer: structure and function. Int J Mol Sci 14, 6259-6305, doi: 10.3390/ijms14036259 (2013).
Bharadwaj, A., Kempster, E. & Waisman, D. M. The Annexin A2/S100A10 Complex: The Mutualistic Symbiosis of Two Distinct Proteins. Biomolecules 11, doi: 10.3390/biom11121849 (2021).
Gerke, V., Creutz, C. E. & Moss, S. E. Annexins: linking Ca2+ signalling to membrane dynamics. Nat Rev Mol Cell Biol 6, 449-461, doi: 10.1038/nrm1661 (2005).
Benowitz, L. I., He, Z. & Goldberg, J. L. Reaching the brain: Advances in optic nerve regeneration. Exp Neurol, doi: 10.1016/j.expneurol.2015.12.015 (2015).
Zhang, J. et al. Silicone oil-induced ocular hypertension and glaucomatous neurodegeneration in mouse. eLife 8, doi: 10.7554/eLife.45881 (2019).
Zhang, J. et al. A Reversible Silicon Oil-Induced Ocular Hypertension Model in Mice. Journal of visualized experiments: JoVE 153, doi: 10.3791/60409 (2019).
Fang, F. et al. Chronic mild and acute severe glaucomatous neurodegeneration derived from silicone oil-induced ocular hypertension. Scientific reports 11, 9052, doi: 10.1038/s41598-021-88690-x (2021).
Balcer, L. J., Miller, D. H., Reingold, S. C. & Cohen, J. A. Vision and vision-related outcome measures in multiple sclerosis. Brain 138, 11-27, doi: 10.1093/brain/awu335 (2015).
Aktas, O., Albrecht, P. & Hartung, H. P. Optic neuritis as a phase 2 paradigm for neuroprotection therapies of multiple sclerosis: update on current trials and perspectives. Current opinion in neurology 29, 199-204, doi: 10.1097/WCO.0000000000000327 (2016).
Costello, F. et al. Quantifying axonal loss after optic neuritis with optical coherence tomography. Annals of neurology 59, 963-969, doi: 10.1002/ana.20851 (2006).
Nakano, N. et al. Longitudinal and simultaneous imaging of retinal ganglion cells and inner retinal layers in a mouse model of glaucoma induced by N-methyl-D-aspartate. Invest Ophthalmol Vis Sci 52, 8754-8762, doi: 10.1167/iovs. 10-6654 (2011).
Guo, L., Normando, E. M., Nizari, S., Lara, D. & Cordeiro, M. F. Tracking longitudinal retinal changes in experimental ocular hypertension using the cSLO and spectral domain-OCT. Invest Ophthalmol Vis Sci 51, 6504-6513, doi: 10.1167/iovs.10-5551 (2010).
Nagata, A., Higashide, T., Ohkubo, S., Takeda, H. & Sugiyama, K. In vivo quantitative evaluation of the rat retinal nerve fiber layer with optical coherence tomography. Invest Ophthalmol Vis Sci 50, 2809-2815, doi: 10.1167/iovs.08-2764 (2009).
Kumar, V. et al. Increased ER Stress After Experimental Ischemic Optic Neuropathy and Improved RGC and Oligodendrocyte Survival After Treatment With Chemical Chaperon. Invest Ophthalmol Vis Sci 60, 1953-1966, doi: 10.1167/iovs. 18-24890 (2019).
Li, L. et al. Longitudinal Morphological and Functional Assessment of RGC Neurodegeneration After Optic Nerve Crush in Mouse. Frontiers in cellular neuroscience 14, 109, doi: 10.3389/fncel.2020.00109 (2020).
Ichhpujani, P., Jindal, A. & Jay Katz, L. Silicone oil induced glaucoma: a review. Graefe's archive for clinical and experimental ophthalmology=Albrecht von Graefes Archiv fur klinische und experimentelle Ophthalmologie 247, 1585-1593, doi: 10.1007/s00417-009-1155-x (2009).
Kornmann, H. L. & Gedde, S. J. Glaucoma management after vitreoretinal surgeries. Curr Opin Ophthalmol 27, 125-131, doi: 10.1097/ICU.0000000000000238 (2016).
Prusky, G. T., Alam, N. M., Beekman, S. & Douglas, R. M. Rapid quantification of adult and developing mouse spatial vision using a virtual optomotor system. Invest Ophthalmol Vis Sci 45, 4611-4616, doi: 10.1167/iovs.04-0541 (2004).
Douglas, R. M. et al. Independent visual threshold measurements in the two eyes of freely moving rats and mice using a virtual-reality optokinetic system. Vis Neurosci 22, 677-684, doi: 10.1017/S0952523805225166 (2005). Douglas, R. M., Neve, A., Quittenbaum, J. P., Alam, N. M. & Prusky, G. T. Perception of visual motion coherence by rats and mice. Vision research 46, 2842-2847, doi: 10.1016/j.visres.2006.02.025 (2006).
Porciatti, V. Electrophysiological assessment of retinal ganglion cell function. Experimental eye research 141, 164-170, doi: 10.1016/j.exer.2015.05.008 (2015).
Chou, T. H., Bohorquez, J., Toft-Nielsen, J., Ozdamar, O. & Porciatti, V. Robust mouse pattern electroretinograms derived simultaneously from each eye using a common snout electrode. Invest Ophthalmol Vis Sci 55, 2469-2475, doi: 10.1167/iovs. 14-13943 (2014).
Giotti, B. et al. Assembly of a parts list of the human mitotic cell cycle machinery. J Mol Cell Biol 11, 703-718, doi: 10.1093/jmcb/mjy063 (2019).
Lee, Y. R., Chen, M. & Pandolfi, P. P. The functions and regulation of the PTEN tumour suppressor: new modes and prospects. Nat Rev Mol Cell Biol 19, 547-562, doi: 10.1038/s41580-018-0015-0 (2018).
Yang, S. G. et al. Strategies to Promote Long-Distance Optic Nerve Regeneration. Frontiers in cellular neuroscience 14, 119, doi: 10.3389/fncel.2020.00119 (2020).
Benowitz, L. I., He, Z. & Goldberg, J. L. Reaching the brain: Advances in optic nerve regeneration. Exp Neurol 287, 365-373, doi: 10.1016/j.expneurol.2015.12.015 (2017).
Gabel, M. et al. Phosphorylation cycling of Annexin A2 Tyr23 is critical for calcium-regulated exocytosis in neuroendocrine cells. Biochimica et biophysica acta. Molecular cell research 1866, 1207-1217, doi: 10.1016/j.bbamcr.2018.12.013 (2019).
Umbrecht-Jenck, E. et al. S100A10-mediated translocation of annexin-A2 to SNARE proteins in adrenergic chromaffin cells undergoing exocytosis. Traffic 11, 958-971, doi: 10.1111/j.1600-0854.2010.01065.x (2010).
Gabel, M. et al. Annexin A2-dependent actin bundling promotes secretory granule docking to the plasma membrane and exocytosis. J Cell Biol 210, 785-800, doi: 10.1083/jcb.201412030 (2015).
Yoon, C. et al. Low-density lipoprotein receptor-related protein 1 (LRP1)-dependent cell signaling promotes axonal regeneration. J Biol Chem 288, 26557-26568, doi: 10.1074/jbc.M113.478552 (2013).
Landowski, L. M. et al. Low-density Lipoprotein Receptor-related Proteins in a Novel Mechanism of Axon Guidance and Peripheral Nerve Regeneration. J Biol Chem 291, 1092-1102, doi: 10.1074/jbc.M115.668996 (2016). Chytla, A. et al. Not Just Another Scaffolding Protein Family: The Multifaceted MPPs. Molecules 25, doi: 10.3390/molecules25214954 (2020).
Podkalicka, J., Biernatowska, A., Olszewska, P., Tabaczar, S. & Sikorski, A. F. The microdomain-organizing protein MPP1 is required for insulin-stimulated activation of H-Ras. Oncotarget 9, 18410-18421, doi: 10.18632/oncotarget.24847 (2018).
O'Donovan, K. J. et al. B-RAF kinase drives developmental axon growth and promotes axon regeneration in the injured mature CNS. J Exp Med 211, 801-814, doi: 10.1084/jem.20131780 (2014).
Zhang, Y. et al. Elevating Growth Factor Responsiveness and Axon Regeneration by Modulating Presynaptic Inputs. Neuron 103, 39-51 e35, doi: 10.1016/j.neuron.2019.04.033 (2019).
Lamort, A. S., Giopanou, I., Psallidas, I. & Stathopoulos, G. T. Osteopontin as a Link between Inflammation and Cancer: The Thorax in the Spotlight. Cells 8, doi: 10.3390/cells8080815 (2019).
Quinta, H. R. et al. Ligand-mediated Galectin-1 endocytosis prevents intraneural H₂O₂ production promoting F-actin dynamics reactivation and axonal re-growth. Exp Neurol 283, 165-178, doi: 10.1016/j.expneurol.2016.06.009 (2016).
Quinta, H. R., Pasquini, J. M., Rabinovich, G. A. & Pasquini, L. A. Glycan-dependent binding of galectin-1 to neuropilin-1 promotes axonal regeneration after spinal cord injury. Cell Death Differ 21, 941-955, doi: 10.1038/cdd.2014.14 (2014).
Higuero, A. M., Diez-Revuelta, N. & Abad-Rodriguez, J. The sugar code in neuronal physiology. Histochem Cell Biol 147, 257-267, doi: 10.1007/s00418-016-1519-3 (2017).
Nio-Kobayashi, J. & Itabashi, T. Galectins and Their Ligand Glycoconjugates in the Central Nervous System Under Physiological and Pathological Conditions. Front Neuroanat 15, 767330, doi: 10.3389/fnana.2021.767330 (2021).
Sakaguchi, M. et al. Regulation of adult neural progenitor cells by Galectin-1/beta1 Integrin interaction. J Neurochem 113, 1516-1524, doi: 10.1111/j.1471-4159.2010.06712.x (2010).
Lin, L., Wu, C. & Hu, K. Tissue Plasminogen Activator Activates NF-κB through a Pathway Involving Annexin A2/CD11b and Integrin-Linked Kinase. Journal of the American Society of Nephrology 23, 1329-1338, doi: 10.1681/asn.2011111123 (2012).
Zhang, C. et al. Coupling of Integrin alpha5 to Annexin A2 by Flow Drives Endothelial Activation. Circulation research 127, 1074-1090, doi: 10.1161/CIRCRESAHA. 120.316857 (2020).
Almasabi, S., Ahmed, A. U., Boyd, R. & Williams, B. R. G. A Potential Role for Integrin-Linked Kinase in Colorectal Cancer Growth and Progression via Regulating Senescence and Immunity. Frontiers in genetics 12, 638558, doi: 10.3389/fgene.2021.638558 (2021).
Persad, S. et al. Inhibition of integrin-linked kinase (ILK) suppresses activation of protein kinase B/Akt and induces cell cycle arrest and apoptosis of PTEN-mutant prostate cancer cells. Proc Natl Acad Sci USA 97, 3207-3212, doi: 10.1073/pnas.060579697 (2000).
Morimoto, A. M. et al. The MMAC1 tumor suppressor phosphatase inhibits phospholipase C and integrin-linked kinase activity. Oncogene 19, 200-209, doi: 10.1038/sj.onc. 1203288 (2000).
Ribeiro, M. et al. Neural Cadherin Plays Distinct Roles for Neuronal Survival and Axon Growth under Different Regenerative Conditions. eNeuro 7, doi: 10.1523/ENEURO.0325-20.2020 (2020).
Bray, E. R. et al. Thrombospondin-1 Mediates Axon Regeneration in Retinal Ganglion Cells. Neuron 103, 642-657 e647, doi: 10.1016/j.neuron.2019.05.044 (2019).
Peterson, S. L. et al. Retinal Ganglion Cell Axon Regeneration Requires Complement and Myeloid Cell Activity within the Optic Nerve. J Neurosci 41, 8508-8531, doi: 10.1523/JNEUROSCI.0555-21.2021 (2021).
Hellal, F. et al. Microtubule stabilization reduces scarring and causes axon regeneration after spinal cord injury. Science 331, 928-931, doi: 10.1126/science.1201148 (2011).
Nawabi, H. et al. Doublecortin-Like Kinases Promote Neuronal Survival and Induce Growth Cone Reformation via Distinct Mechanisms. Neuron 88, 704-719, doi: 10.1016/j.neuron.2015.10.005 (2015).
Wang, X. W. et al. Knocking Out Non-muscle Myosin II in Retinal Ganglion Cells Promotes Long-Distance Optic Nerve Regeneration. Cell reports 31, 107537, doi: 10.1016/j.celrep.2020.107537 (2020).
Brasaemle, D. L. Thematic review series: adipocyte biology. The perilipin family of structural lipid droplet proteins: stabilization of lipid droplets and control of lipolysis. J Lipid Res 48, 2547-2559, doi: 10.1194/jlr.R700014-JLR200 (2007).
Yang, C. et al. Rewiring Neuronal Glycerolipid Metabolism Determines the Extent of Axon Regeneration. Neuron 105, 276-292 e275, doi: 10.1016/j.neuron.2019.10.009 (2020).
Chauhan, M. Z. et al. Multi-Omic Analyses of Growth Cones at Different Developmental Stages Provides Insight into Pathways in Adult Neuroregeneration. iScience 23, 100836, doi: 10.1016/j.isci.2020.100836 (2020).
Pertea, M., Kim, D., Pertea, G. M., Leek, J. T. & Salzberg, S. L. Transcript-level expression analysis of RNA-seq experiments with HISAT, String Tie and Ballgown. Nature protocols 11, 1650-1667, doi: 10.1038/nprot.2016.095 (2016).
Kim, D., Paggi, J. M., Park, C., Bennett, C. & Salzberg, S. L. Graph-based genome alignment and genotyping with HISAT2 and HISAT-genotype. Nature biotechnology 37, 907-915, doi: 10.1038/s41587-019-0201-4 (2019).
Hao, Y. et al. Integrated analysis of multimodal single-cell data. Cell 184, 3573-3587 e3529, doi: 10.1016/j.cell.2021.04.048 (2021).
Yu, G., Wang, L. G., Han, Y. & He, Q. Y. clusterProfiler: an R package for comparing biological themes among gene clusters. OMICS 16, 284-287, doi: 10.1089/omi.2011.0118 (2012).
Renier, N. et al. iDISCO: a simple, rapid method to immunolabel large tissue samples for volume imaging. Cell 159, 896-910 (2014).
Leon, S., Yin, Y., Nguyen, J., Irwin, N. & Benowitz, L. I. Lens injury stimulates axon regeneration in the mature rat optic nerve. J Neurosci 20, 4615-4626 (2000).
Karl, M. O. et al. Stimulation of neural regeneration in the mouse retina. Proc Natl Acad Sci USA 105, 19508-19513, doi: 10.1073/pnas.0807453105 (2008).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of promoting axon regeneration and neuroprotection in a mammal, the method comprising:
contacting the neuronal cell body with an effective dose of a regeneration associated gene (RAG) agent to promote axon regeneration and neuroprotection of the neuronal cell body and axon.

2. The method of claim 1, wherein the RAG is one or more of ANXA2 (Annexin A2), tPA (tissue plasminogen activator), GSN (gelsolin), VIM (Vimentin), MPP1 (Membrane Palmitoylated Protein 1), ILK (Integrin Linked Kinase), ECM1 (extracellular matrix protein 1), CALM1 (calmodulin 1), AND ACAA2 (Acetyl-CoA Acyltransferase 2).

3. The method of claim 1, wherein the RAG is ANXA2 in combination with TPA.

4. The method of claim 1, wherein the RAG agent comprises a gene therapy vector.

5. The method of claim 4, wherein the vector is a mammalian AAV vector.

6. The method of claim 4, wherein the vector comprises a RAG coding sequence operably linked to a promoter active in retinal ganglion cells (RGCs).

7. The method of claim 1, wherein the axonopathy is an optic nerve (ON) neuropathy.

8. The method of claim 7, wherein the ON neuropathy is retinal ganglion cell and ON degeneration, including glaucoma, optic neuritis, ON traumatic injury and other ON-related diseases.

9. The method of claim 8, wherein the ON neuropathy is glaucoma.

10. The method of claim 1, wherein the subject is human.

11. The method of claim 1, wherein the RAG agent is intravitreally administered.

12. A composition comprising:
a mammalian viral vector, which comprises:
a promoter, or functional fragment thereof, that promotes expression of an operably linked coding sequence specifically in retinal ganglion cells (RGCs), and
a sequence encoding a functional human RAG protein, or a variant thereof.

13. The vector of claim 12, wherein the vector is a mammalian AAV vector.

14. The vector of claim 12, wherein the RAG is one or more of ANXA2, TPA, GSN, VIM, MPP1, ILK, ECM1, CALM1, AND ACAA2.

15. The vector of claim 12, wherein the RAG is ANXA2 in combination with TPA.

16. The vector of claim 12, wherein the promoter is a murine Sncg promoter.

17. An AAV virus particle comprising a vector of claim 12.
